(19) United States
(12) Patent Application Publication
(10) Pub. No.: US 2020/0255436 A1
(43) Pub. Date: Aug. 13, 2020

(54) CORTICOTROPIN RELEASING FACTOR RECEPTOR ANTAGONISTS

(71) Applicant: Spruce Biosciences, Inc., San Francisco, CA (US)

(72) Inventors: Alexis HOWERTON, San Francisco, CA (US); Hal GERBER, San Francisco, CA (US); Sami KARABORNI, San Francisco, CA (US); Sangita GHOSH, San Francisco, CA (US)

(21) Appl. No.: 16/788,121

(22) Filed: Feb. 11, 2020

Related U.S. Application Data
(60) Provisional application No. 62/804,646, filed on Feb. 12, 2019.

(51) Int. Cl. C07D 487/04; C07D 417/04; A61K 9/20

(52) U.S. Cl. CPC C07D 487/04; A61K 9/20; C07D 417/04

(57) ABSTRACT

The present disclosure provides forms of CRF antagonists and methods using such CRF antagonists for treating diseases.

### CROSS-REFERENCE

This application claims the benefit of U.S. Provisional Patent Application No. 62/804,646, filed Feb. 12, 2019, which is entirely incorporated herein by reference.

### BACKGROUND

Corticotropin releasing factor (CRF) is a 41 amino acid peptide that is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain. There is also evidence that CRF plays a significant role in integrating the response in the immune system to physiological, psychological, and immunological stressors.

### SUMMARY

The present disclosure provides forms of CRF antagonists and methods using such CRF antagonists for treating diseases. In some embodiments, such CRF antagonists are used to treat congenital adrenal hyperplasia (CAH).

In one aspect, the present disclosure provides a compound of Formula I:

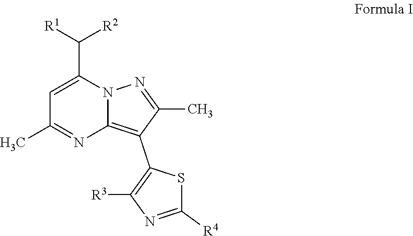

or a pharmaceutically acceptable salt thereof, wherein
R¹ and R² are independently ethyl or n-propyl;

R³ is H, Cl, Br, methyl, trifluoromethyl or methoxy;

R⁴ is H, Br, R^{a}R^{b}N-, methoxymethyl, n-butyl, acetamido, pyridin-4-yl, morpholin-4-yl,

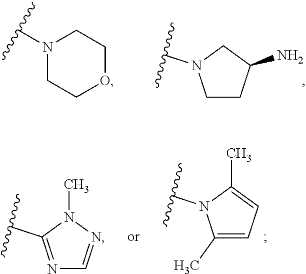

R^{a} and R^{b} are independently hydrogen, C₁-C₃ alkyl, H₂NCH₂CH₂—,

(CH₃)₃COC(O)NHCH₂CH₂—, or CH₃CH₂CH₂NHCH₂CH₂;
wherein the compound of Formula I contains at least one deuterium atom; and
wherein the deuterium enrichment content of the compound of Formula I is at least about 1%.

In one embodiment, R³ is Cl, Br, methyl or trifluoromethyl. In one embodiment, R³ is Cl or Br. In one embodiment, R⁴ is R^{a}R^{b}N-, pyridin-4-yl, morpholin-4-yl, or

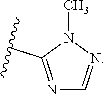 [SMILES: *c1ncnn1C]

In one embodiment, R⁴ is morpholin-4-yl or

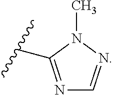 [SMILES: *c1ncnn1C]

In one embodiment, R4 is R^{a}R^{b}N— and R^{a} and R^{b} are independently C₁-C₃alkyl.

In one embodiment, the deuterium enrichment in the compound of Formula I is at least about 2%. In one embodiment, the deuterium enrichment in the compound of Formula I is at least about 3%. In one embodiment, the deuterium enrichment in the compound of Formula I is at least about 4%. In one embodiment, the deuterium enrichment in the compound of Formula I is at least about 5%. In one embodiment, the deuterium enrichment in the compound of Formula I is at least about 10%.

In one embodiment, the present disclosure provides 3-[4-bromo-2-(2-methyl-2H-[1,2,4]triazol-3-yl)-thiazol-5-yl]-2,5-dimethyl-7-(1-propyl-butyl)-pyrazolo[1,5-a]pyrimidine, wherein the 7-(1-propyl-butyl)moiety comprises at least one deuterium.

In one embodiment, the present disclosure provides 3-(4-bromo-2-(2-methyl-2H-[1,2,4]triazol-3-yl)-thiazol-5-yl)-7-(1-ethyl-propyl)-2,5-dimethyl-pyrazolo[1,5-a]pyrimidine, wherein the 7-(1-ethyl-propyl) moiety comprises at least one deuterium.

In one embodiment, the present disclosure provides 3-(4-chloro-2-morpholin-4-yl-thiazol-5-yl)-7-(1-ethyl-propyl)-2,5-dimethy-1-pyrazolo[1,5-a]pyrimidine wherein the 7-(1-ethyl-propyl) moiety comprises at least one deuterium.

In one embodiment, the present disclosure provides 3-(4-chloro-2-(morpholin-4-yl)thiazol-5-yl)-7-(1-ethyl-propyl)-2,5-dimethylpyrazolo(1,5-a)pyrimidine or 4-(4-chloro-5-(2,5-dimethyl-7-(pentan-3-yl)pyrazolo[1,5-a]pyrimidin-3-yl)thiazol-2-yl)morpholine, wherein the 7-(1-ethyl-propyl) moiety comprises at least one deuterium.

In one aspect, the present disclosure provides a pharmaceutical composition comprising Compound 1:

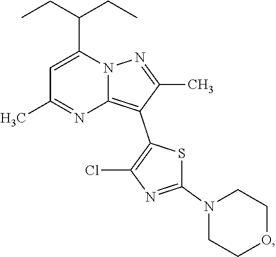 [SMILES: CCC(CC)c1cc(C)nc2c(-c3sc(N4CCOCC4)nc3Cl)c(C)nn12]

or a pharmaceutically acceptable salt or solvate thereof, and Compound 2:

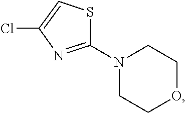 [SMILES: Clc1csc(N2CCOCC2)n1]

or a pharmaceutically acceptable salt or solvate thereof.

In one embodiment, the pharmaceutical composition comprises from about 0.5 ppm to about 5000 ppm of Compound 2, or a pharmaceutically acceptable salt or solvate thereof the pharmaceutical composition comprises from about 0.00005 weight % to about 0.5 weight % of Compound 2, or a pharmaceutically acceptable salt or solvate thereof.

In one aspect, the present disclosure provides a pharmaceutical composition comprising Compound 1:

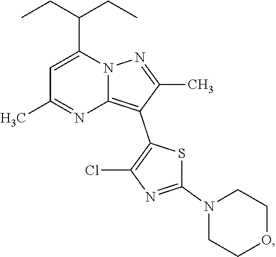 [SMILES: CCC(CC)c1cc(C)nc2c(-c3sc(N4CCOCC4)nc3Cl)c(C)nn12]

or a pharmaceutically acceptable salt or solvate thereof, which has a D₉₀ from about 1 μm to about 20 μm.

In one embodiment, Compound 1, or a pharmaceutically acceptable salt or solvate thereof has a D₉₀ from about 5 μm to about 15 μm. In one embodiment, Compound 1, or a pharmaceutically acceptable salt or solvate thereof has a D₉₀ of about 6 μm. In one embodiment, Compound 1, or a pharmaceutically acceptable salt or solvate thereof has a D₉₀ of about 7 μm. In one embodiment, Compound 1, or a pharmaceutically acceptable salt or solvate thereof has a D₉₀ of about 8 μm. In one embodiment, Compound 1, or a pharmaceutically acceptable salt or solvate thereof has a D₉₀ of about 9 μm. In one embodiment, Compound 1, or a pharmaceutically acceptable salt or solvate thereof has a D₉₀ of about 10 μm. In one embodiment, Compound 1, or a pharmaceutically acceptable salt or solvate thereof a D₉₀ of about 11 μm.

In one embodiment, the pharmaceutical composition is in an oral dosage form. In one embodiment, the oral dosage form is selected from the group consisting of a tablet, a capsule, a buccal tablet, a sub-lingual table, an orally-disintegrating tablet, a thin film, a liquid solution, a liquid suspension, a syrup, a powder, solid crystals, minitabs, coated pellets and sachets.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Compound 1 in patients with CAH following 14-days of once daily dosing at each level;

FIG. 2 demonstrates the attenuation of ACTH across different subjects due to the administration of Compound 1;

FIG. 3 demonstrates the reduction in 17-OHP due to the administration of Compound 1;

FIG. 4 demonstrates the reduction of Androstenedione due to the administration of Compound 1;

FIG. 5 demonstrates the percentage release of Manufacturing Formulae A-1, A-2 and A-3 in 0.1N HCL+1.0% SDS, media at 50 rpm and 900 mL using USP-II (Paddle);

FIG. 6 demonstrates the percentage release of Manufacturing Formulae B-1, B-2 and B-3 in 0.1N HCL+1.0% SDS, media at 50 rpm and 900 mL using USP-II (Paddle);

FIG. 7 demonstrates the percentage release of Manufacturing Formulae C-1, C-2 and C-3 in 0.1N HCL+1.0% SDS, media at 50 rpm and 900 mL using USP-II (Paddle);

FIG. 8 demonstrates the percentage release of Manufacturing Formula F-1 at different hardness in 0.1N HCL+1.0% SDS, media at 50 rpm and 900 mL using USP-II (Paddle); and

FIG. 9 demonstrates the percentage release of Manufacturing G-1 at different hardness in 0.1N HCL+1.0% SDS, media at 50 rpm and 900 mL using USP-II (Paddle).

### DETAILED DESCRIPTION

CRF has been implicated in psychiatric disorders and neurological diseases including depression and anxiety, as well as the following: Alzheimer's disease, Huntington's disease, progressive supranuclear palsy, amyotrophic lateral sclerosis, Parkinson's disease, epilepsy, migraine, alcohol and substance abuse and associated withdrawal symptoms, obesity, metabolic syndrome, congenital adrenal hyperplasia (CAH), Cushing's disease, hypertension, stroke, irritable bowel syndrome, stress-induced gastric ulceration, premenstrual syndrome, sexual dysfunction, premature labor, inflammatory disorders, allergies, multiple sclerosis, visceral pain, sleep disorders, pituitary tumors or ectopic pituitary derived tumors, chronic fatigue syndrome, and fibromyalgia.

CRF receptor subtypes, CRF1 and CRF2, have been identified and are distributed heterogeneously within the brain thereby suggesting potential functional diversity. For example, widely distributed brain CRF1 receptors are strongly implicated in emotionality accompanying exposure to environmental stressors. Significantly, CRF1, not CRF2, receptors appear to mediate select anxiogenic like behaviors. A more discrete septallhypothalmic distribution and the availability of alternative endogenous ligands suggest a different functional role for the CRF2 receptor. For example, a novel CRF-family neuropeptide with preferential affinity for CRF2 relative to CRF 1 receptors is reported to suppress appetite without producing the profile of behavioral activation observed with selective CRF1 agonism. In other cases, CRF2 agonism produces similar effects to those reported for CRF 1 antagonists or CRF 1 gene deletion. For example, while CRF2 agonists have been proposed as antiobesity agents, CRF1 antagonists may be an important treatment for obesity as well.

Treatment of CAH is based on normalization of hormone and steroid levels using a variety of medications from diagnosis in infancy through adulthood. Glucocorticoids are the current standard treatment in CAH and are used both to correct the endogenous Cortisol deficiency and for reducing the elevated ACTH levels from the pituitary, which drives increased androgen production. Unlike the treatment of Addison's disease (adrenal insufficiency), in which Cortisol replacement is sufficient, the treatment of CAH must also reduce ACTH production, to control the subsequent androgen excess as well. Thus, the goals of glucocorticoid treatment include Cortisol replacement and suppression of ACTH to prevent virilization and menstrual disturbances in women. Mineralocorticoid replacement is needed to achieve normal plasma renin activity for maintenance of regular blood pressure, electrolyte balance, and volume status in those patients with the salt-wasting form of CAH.

The regimen of glucocorticoid treatment must support normal physiology and also ensure that sufficient Cortisol is available during events that may elicit a strong stress response (e.g., intercurrent illness, exercise, hypotension). Careful monitoring is also necessary to avoid the development of Addisonian syndrome due to under-treatment. Overtreatment with mineralocorticoids may cause hypertension while under-treatment may lead to low blood pressure, salt loss, fatigue and increased requirements for glucocorticoids. Typical laboratory tests for monitoring treatment efficacy include measurement of plasma concentrations of 17-OHP, androstenedione, testosterone, renin activity, and electrolytes.

Adult patients with CAH have an increased prevalence of risk factors for cardiovascular disease including obesity, hypertension, and insulin resistance. A study of a large cohort of pediatric and adult CAH patients (n=244) demonstrated that patients are prescribed a variety of glucocorticoid treatment regimens yet frequently suffer from poor hormonal control and the aforementioned adverse outcomes. Treatment of CAH includes efforts to normalize the Cortisol deficiency with glucocorticoids (usually hydrocortisone in children but often more potent agents with narrow therapeutic indices, such as dexamethasone, in adults) and, if necessary for salt-wasting, mineralocorticoids (usually fludrocortisone). The glucocorticoid doses required to achieve sufficient suppression of excess androgens, however, are usually well above the normal physiologic dose used for Cortisol replacement alone as in patients with Addison's disease. This increased exposure to glucocorticoids can lead to increased cardiovascular risk factors, glucose intolerance, and decreased bone mineral density in CAH patients.

CRF is believed to be the major physiological regulator of the basal and stress-induced release of adrenocorticotropic hormone (“ACTH”), β-endorphin, and other proopiomelanocortin (“POMC”)-derived peptides from the anterior pituitary. Secretion of CRF causes release of ACTH from corticotrophs in the anterior pituitary via binding to the CRF₁ receptor, a member of the class B family of G-protein coupled receptors.

Due to the physiological significance of CRF₁, the development of biologically-active small molecules having significant CRF receptor binding activity and which are capable of antagonizing the CRF₁ receptor remains a desirable goal and has been the subject of ongoing research and development for the treatment of anxiety, depression, irritable bowel syndrome, post-traumatic stress disorder, and substance abuse.

The pituitary hormone ACTH, under the control of hypothalamic corticotropin-releasing factor (CRF), stimulates uptake of cholesterol and drives the synthesis of pregnenolone initiating steroidogenesis in the adrenal gland. The adrenal cortex is comprised of three zones, which produce distinct classes of hormones many of which are driven by ACTH mobilizing cholesterol through this pathway. Deficiencies in these enzymes as a result of mutation or deletion cause the substrate concentrations to increase. In the most common form of CAH resulting from mutations or deletions in the 21-hydroxylase gene (CYP21A2), potent androgens are produced by the adrenal because of the accumulation of the steroid precursors, progesterone and 17-hydroxyprogesterone (17-OHP). Plasma levels of 17-OHP can reach 10-1000 times the normal concentration in these cases. These increases result in the overproduction of androgens, specifically androstenedione, testosterone, and dihydroxytestosterone causing virilization in females. In addition, 21-hydroxylase deficiency in CAH causes insufficient biosynthesis of glucocorticoids and mineralocorticoids, specifically Cortisol and aldosterone. Cortisol is a critical negative feedback regulator of hypothalamic CRF secretion and pituitary ACTH release. The lack of glucocorticoid synthesis and release eliminates the restraint on the hypothalamus and pituitary, which causes ACTH levels to increase. The excessive ACTH stimulation causes hypertrophy of the zona fasciculata and zona reticularis resulting in adrenal hyperplasia.

In one embodiment, the CRF receptor antagonist useful for the treatment of CAH is 3-(4-Chloro-2-(morpholin-4-yl) thiazol-5-yl)-7-(1-ethylpropyl)-2,5-dimethylpyrazolo(1,5-a)pyrimidine.

### Certain Definitions

Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein and in the appended claims, the singular forms “a,” “an,” and “the” include plural reference unless the context clearly dictates otherwise. Thus, for example, reference to “an excipient” is a reference to one or more excipients and equivalents thereof known to those skilled in the art, and so forth.

The term “about” is used to indicate that a value includes the standard level of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and to “and/or.”

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “alkyl”, as used herein, means a saturated aliphatic hydrocarbon group, which may be straight or branched, having 1 to 5 carbon atoms in the chain.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. “Administering” a pharmaceutical composition may be accomplished by injection, topical administration, and oral administration or by other methods alone or in combination with other known techniques.

By “pharmaceutically acceptable”, it is meant the carrier, diluent or excipient must be compatible with the other ingredients of the composition and not deleterious to the recipient thereof.

The term “pharmaceutical composition” means a composition comprising at least one active ingredient, such as Compound 1, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “supraphysiologic” amount” describes hormones levels that are elevated compared to average levels found in healthy individuals.

The term “physiologic amount” describes average hormone levels found in healthy individuals.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Compounds

Disclosed herein is a compound of Formula I:

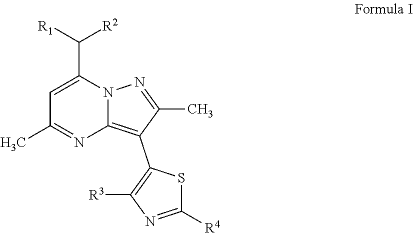

or a pharmaceutically acceptable salt thereof, wherein

R¹ and R² are independently ethyl or n-propyl;

R³ is H, Cl, Br, methyl, trifluoromethyl or methoxy;

R⁴ is H, Br, R^{a}R^{b}N—, methoxymethyl, n-butyl, acetamido, pyridin-4-yl, morpholin-4-yl,

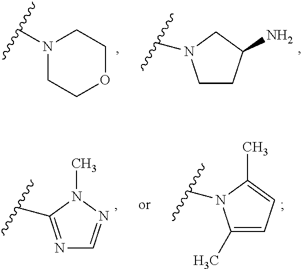

R^{a} and R^{b} are independently hydrogen, C₁-C₃ alkyl, H₂NCH₂CH₂—,

(CH₃)₃COC(O)NHCH₂CH₂—, or CH₃CH₂CH₂NHCH₂CH₂;
wherein the compound of Formula I contains at least one deuterium atom; and
wherein the deuterium enrichment in the compound of Formula I is at least about 1%.

In some embodiments, R³ is Cl, Br, methyl or trifluoromethyl. In one embodiment, R³ is Cl or Br. In some embodiments, R⁴ is R^{a}R^{b}N—, pyridin-4-yl, morpholin-4-yl, or

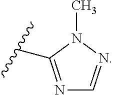 [SMILES: *c1ncnn1C]

In some embodiments, R⁴ is morpholin-4-yl or

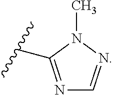 [SMILES: *c1ncnn1C]

In some embodiments, R⁴ is R^{a}R^{b}N— and R^{a} and R^{b} are independently C₁-C₃alkyl.

Disclosed herein is a compound of Formula II:

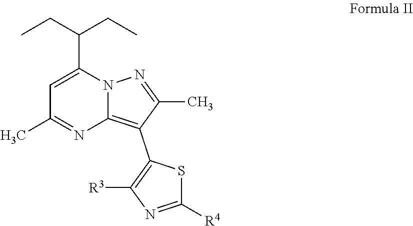

or a pharmaceutically acceptable salt thereof, wherein

R³ is H, Cl, Br, methyl, trifluoromethyl or methoxy;

R⁴ is H, Br, R^{a}R^{b}N—, methoxymethyl, n-butyl, acetamido, pyridin-4-yl, morpholin-4-yl,

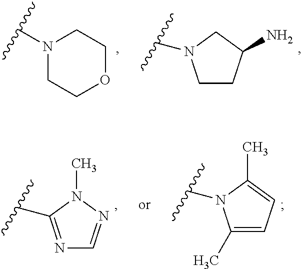

R^{a} and R^{b} are independently hydrogen, C₁-C₃ alkyl, H₂NCH₂CH₂—,

(CH₃)₃COC(O)NHCH₂CH₂—, or CH₃CH₂CH₂NHCH₂CH₂;
wherein the compound of Formula I contains at least one deuterium atom; and
wherein the deuterium enrichment in the compound of Formula I is at least about 1%.

Disclosed herein is a compound of Formula III:

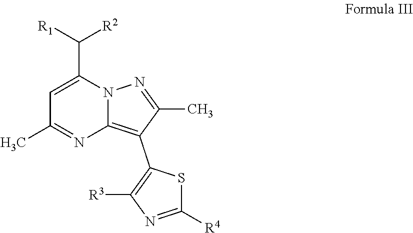

or a pharmaceutically acceptable salt thereof, wherein

R¹ and R² are n-propyl;

R³ is H, Cl, Br, methyl, trifluoromethyl or methoxy;

R⁴ is H, Br, R^{a}R^{b}N—, methoxymethyl, n-butyl, acetamido, pyridin-4-yl, morpholin-4-yl,

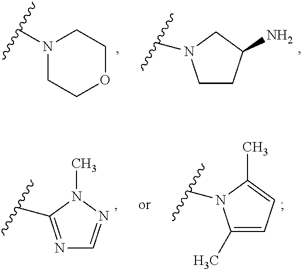

R^{a} and R^{b} are independently hydrogen, C₁-C₃ alkyl, H₂NCH₂CH₂—,

(CH₃)₃COC(O)NHCH₂CH₂—, or CH₃CH₂CH₂NHCH₂CH₂;
wherein the compound of Formula I contains at least one deuterium atom; and
wherein the deuterium enrichment in the compound of Formula I is at least about 1%.

Disclosed herein is a compound of Formula IV:

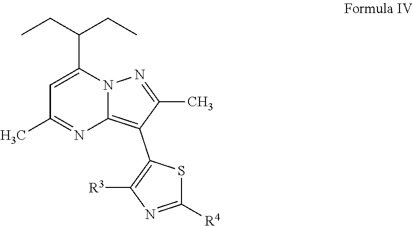

or a pharmaceutically acceptable salt thereof, wherein

R³ is Cl, Br, methyl, trifluoromethyl or methoxy;

R⁴ is H, Br, R^{a}R^{b}N—, methoxymethyl, n-butyl, acetamido, pyridin-4-yl, morpholin-4-yl,

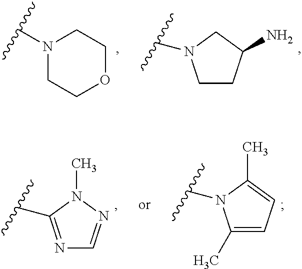

R^{a} and R^{b} are independently hydrogen, C₁-C₃ alkyl, H₂NCH₂CH₂—,

(CH₃)₃COC(O)NHCH₂CH₂—, or CH₃CH₂CH₂NHCH₂CH₂;
wherein the compound of Formula I contains at least one deuterium atom; and
wherein the deuterium enrichment in the compound of Formula I is at least about 1%.

In one embodiment, R³ is Cl, Br, methyl, or methoxy. In one embodiment, R³ is Cl, Br, or methyl. In one embodiment, R³ is Cl or Br. In one embodiment, R³ is Cl. In one embodiment, R³ is Br.

In one embodiment, R⁴ is Br, R^{a}R^{b}N-, methoxymethyl, n-butyl, acetamido, pyridin-4-yl, morpholin-4-yl,

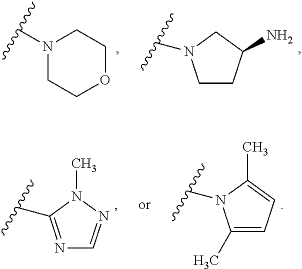

In one embodiment, R⁴ is Br, methoxymethyl, n-butyl, acetamido, pyridin-4-yl, morpholin-4-yl,

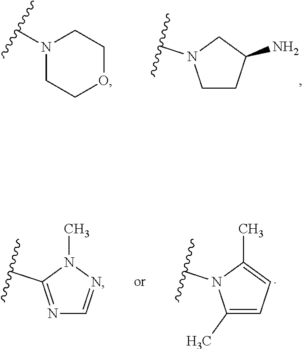

In one embodiment, R⁴ is Br, n-butyl, acetamido, pyridin-4-yl, morpholin-4-yl,

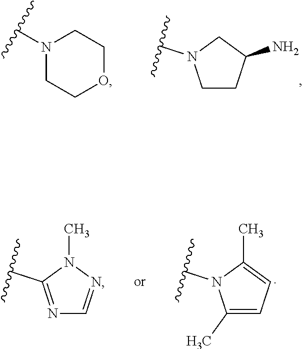

In one embodiment, R⁴ is Br, acetamido, pyridin-4-yl, morpholin-4-yl,

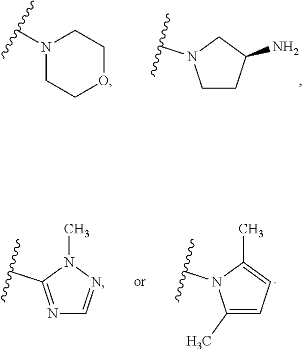

In one embodiment, R⁴ is Br, pyridin-4-yl, morpholin-4-yl,

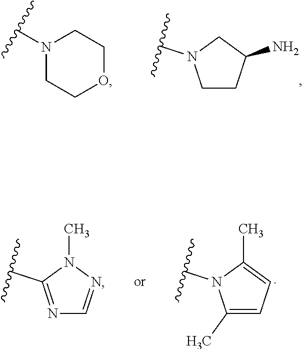

In one embodiment, R⁴ is pyridin-4-yl, morpholin-4-yl,

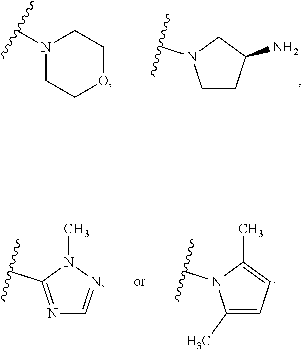

In one embodiment, R⁴ is morpholin-4-yl,

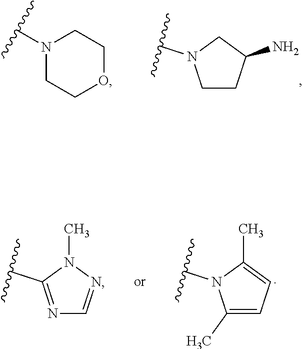

In one embodiment, R⁴ is

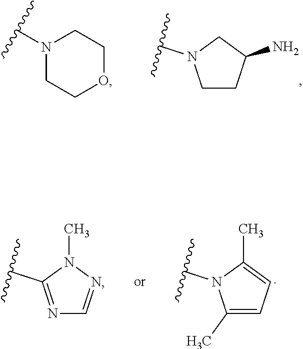

In one embodiment, R⁴ is

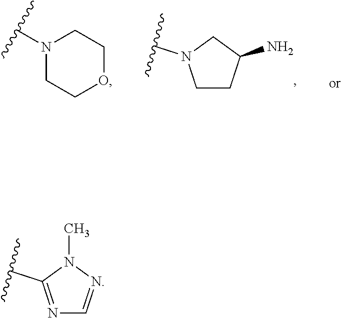

In one embodiment, R⁴ is

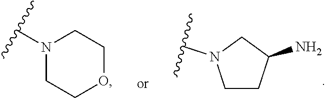 [SMILES: *N1CCOCC1 ; "or" ; *N1CC[C@H](N)C1 ; "."]

In one embodiment, R⁴ is

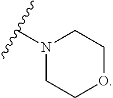 [SMILES: *N1CCOCC1]

In one embodiment, the deuterium enrichment content of a compound of Formula I, II, III, or IV is at least about 1%, at least about 2%, at least about 3%, at least about 4%, at least about 5%, at least about 6%, at least about 7%, at least about 8%, at least about 9%, or at least about 10%, less than about 100%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, from about 1% to about 100%, from about 2% to about 100%, from about 3% to about 100%, from about 4% to about 100%, from about 5% to about 100%, from about 6% to about 100%, from about 7% to about 100%, from about 8% to about 100%, from about 9% to about 100%, from about 10% to about 100%, from about 1% to about 90%, from about 1% to about 80%, from about 1% to about 70%, from about 1% to about 60%, from about 1% to about 50%, from about 1% to about 40%, from about 1% to about 30%, from about 1% to about 20%, or from about 1% to about 10%.

Disclosed herein is 3-(4-Chloro-2-(morpholin-4-yl)thiazol-5-yl)-7-(1-ethylpropyl)-2,5-dimethylpyrazolo(1,5-a)pyrimidine (or alternatively 4-(4-chloro-5-(2,5-dimethyl-7-(pentan-3-yl)pyrazolo[1,5-a]pyrimidin-3-yl)thiazol-2-yl)morpholine), a pharmaceutically acceptable salt, and/or a solvate thereof:

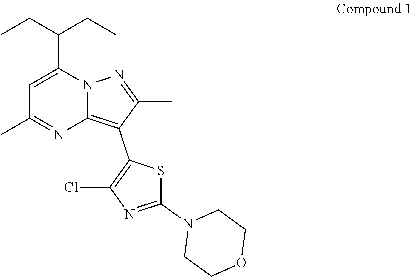 [SMILES: CCC(CC)c1cc(C)nc2c(-c3sc(N4CCOCC4)nc3Cl)c(C)nn12]

In some embodiments, 4-(4-chloro-5-(2,5-dimethyl-7-(pentan-3-yl)pyrazolo[1,5-a]pyrimidin-3-yl)thiazol-2-yl)morpholine is referred to as Compound 1. In some embodiments, 3-(4-Chloro-2-(morpholin-4-yl)thiazol-5-yl)-7-(1-ethylpropyl)-2,5-dimethylpyrazolo(1,5-a)pyrimidine is referred to as Compound 1. In some embodiments, Compound 1 contains at least one deuterium atom; and the deuterium enrichment in Compound 1 is at least about 1%.

In one embodiment, the deuterium enrichment content of Compound 1 is at least about 1%, at least about 2%, at least about 3%, at least about 4%, at least about 5%, at least about 6%, at least about 7%, at least about 8%, at least about 9%, or at least about 10%, less than about 100%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, from about 1% to about 100%, from about 2% to about 100%, from about 3% to about 100%, from about 4% to about 100%, from about 5% to about 100%, from about 6% to about 100%, from about 7% to about 100%, from about 8% to about 100%, from about 9% to about 100%, from about 10% to about 100%, from about 1% to about 90%, from about 1% to about 80%, from about 1% to about 70%, from about 1% to about 60%, from about 1% to about 50%, from about 1% to about 40%, from about 1% to about 30%, from about 1% to about 20%, or from about 1% to about 10%.

Disclosed herein is 3-[4-bromo-2-(2-methyl-2H[1,2,4]triazol-3-yl)-thiazol-5-yl]-2,5-dimethyl-7-(1-propyl-butyl)-pyrazolo[1,5-a]pyrimidine (or alternatively, 3-(4-bromo-2-(2-methyl-2H-[1,2,4]triazol-3-yl)-thiazol-5-yl)-7-(1-ethyl-propyl)-2,5-dimethyl-pyrazolo[1,5-a]pyrimidine) , a pharmaceutically acceptable salt, and/or solvate thereof.

In one embodiment, the 7-(1-propyl-butyl) moiety comprises 1 deuterium, 2 deuterium, 3 deuterium, 4 deuterium, 5 deuterium, 6 deuterium, 7 deuterium, 8 deuterium, 9 deuterium, 10 deuterium, 11 deuterium, 12 deuterium, 13 deuterium, 14 deuterium, or 15 deuterium. In one embodiment, the 7-(1-propyl-butyl) moiety comprises 3 deuterium, 6 deuterium, 8 deuterium, 10 deuterium, 12 deuterium, 14 deuterium, or 15 deuterium. In one embodiment, one or both of the 2,5-dimethyl groups independent comprises 1 deuterium, 2 deuterium, 3 deuterium, 4 deuterium, 5 deuterium, or 6 deuterium. In one embodiment, one or both of the 2,5-dimethyl groups independent comprises 3 deuterium, or 6 deuterium. In one embodiment, the mopholino moiety comprises 1 deuterium, 2 deuterium, 3 deuterium, 4 deuterium, 5 deuterium, 6 deuterium, 7 deuterium, or 8 deuterium. In one embodiment, the mopholino moiety comprises 2 deuterium, 4 deuterium, 6 deuterium, or 8 deuterium.

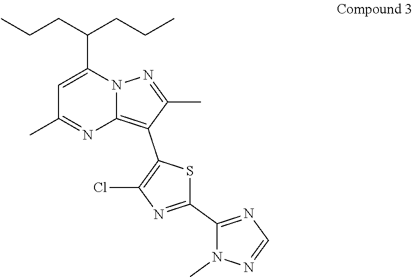 [SMILES: CCCC(CCC)c1cc(C)nc2c(-c3sc(-c4ncnn4C)nc3Cl)c(C)nn12]

In some embodiments, 3-[4-bromo-2-(2-methyl-2H-[1,2,4]triazol-3-yl)-thiazol-5-yl]-2,5-dimethyl-7-(1-propyl-butyl)-pyrazolo[1,5-a]pyrimidine is referred to as Compound 2. In some embodiments, 3-(4-bromo-2-(2-methyl-2H-[1,2,4]triazol-3-yl)-thiazol-5-yl)-7-(1-ethyl-propyl)-2,5-dimethyl-pyrazolo[1,5-a]pyrimidine is referred to as Compound 3. In some embodiments, Compound 2 contains at least one deuterium atom; and the deuterium enrichment in Compound 2 is at least about 1%.

In one embodiment, the deuterium enrichment content of Compound 3 is at least about 1%, at least about 2%, at least about 3%, at least about 4%, at least about 5%, at least about 6%, at least about 7%, at least about 8%, at least about 9%, or at least about 10%, less than about 100%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, from about 1% to about 100%, from about 2% to about 100%, from about 3% to about 100%, from about 4% to about 100%, from about 5% to about 100%, from about 6% to about 100%, from about 7% to about 100%, from about 8% to about 100%, from about 9% to about 100%, from about 10% to about 100%, from about 1% to about 90%, from about 1% to about 80%, from about 1% to about 70%, from about 1% to about 60%, from about 1% to about 50%, from about 1% to about 40%, from about 1% to about 30%, from about 1% to about 20%, or from about 1% to about 10%.

In one embodiment, the 7-(1-ethyl-propyl) moiety comprises 1 deuterium, 2 deuterium, 3 deuterium, 4 deuterium, 5 deuterium, 6 deuterium, 7 deuterium, 8 deuterium, 9 deuterium, 10 deuterium, or 11 deuterium. In one embodiment, the 7-(1-propyl-butyl) moiety comprises 3 deuterium, 6 deuterium, 8 deuterium, 10 deuterium, or 11 deuterium. In one embodiment, one or both of the 2,5-dimethyl groups independent comprises 1 deuterium, 2 deuterium, 3 deuterium, 4 deuterium, 5 deuterium, or 6 deuterium. In one embodiment, one or both of the 2,5-dimethyl groups independent comprises 3 deuterium, or 6 deuterium. In one embodiment, the mopholino moiety comprises 1 deuterium, 2 deuterium, 3 deuterium, 4 deuterium, 5 deuterium, 6 deuterium, 7 deuterium, or 8 deuterium. In one embodiment, the mopholino moiety comprises 2 deuterium, 4 deuterium, 6 deuterium, or 8 deuterium.

### Pharmaceutical Compositions

Poorly soluble drugs may be difficult to formulate using technologies such as high shear wet granulation. Optimum delivery of poorly soluble drugs may require complex technologies such as solid solutions or amorphous dispersions (for example hot melt extrusion or spray drying), nano-formulations or lipid-based formulations. Hydrophobic drug substances which may be considered poorly soluble according to USP criteria may also be difficult to granulate with water and other excipients as most excipients for immediate-release formulations may be water soluble or water-swellable.

Making a small tablet of a high dose drug substance that is poorly soluble may require a high concentration of the drug substance. However, as the drug concentration is increased above a certain level, formation of granules may become more difficult, and at a certain drug load, it may become impossible.

In one embodiment, the pharmaceutical compositions described herein may be for a pediatric population. Hence, it may be necessary to maintain the pharmaceutical composition as small as possible to facilitate swallowing of pills and therefore increase patient compliance. In some embodiments, tablet weights are less than 400 mg. In some embodiments, tablet weights are less than 300 mg. In some embodiments, where dose strength is 200 mg, the drug load in the tablet is higher than 50%. In some embodiments, where dose strength is 200 mg, the drug load in the tablet is higher than 66%. In some embodiments, where dose strength is 200 mg, the drug load is as high as possible.

Disclosed herein is a pharmaceutical composition comprising Compound 1, a pharmaceutically acceptable salt, and/or a solvate thereof.

Disclosed herein is a pharmaceutical composition comprising Compound 1:

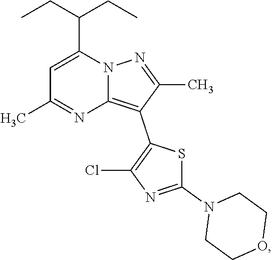 [SMILES: CCC(CC)c1cc(C)nc2c(-c3sc(N4CCOCC4)nc3Cl)c(C)nn12]

or a pharmaceutically acceptable salt or solvate thereof, and Compound 2:

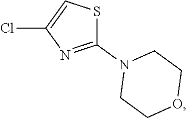 [SMILES: Clc1csc(N2CCOCC2)n1]

or a pharmaceutically acceptable salt or solvate thereof.

In one embodiment, the pharmaceutical composition comprises from about 0.5 ppm to about 5000 ppm of Compound 2, or a pharmaceutically acceptable salt or solvate thereof. In one embodiment, the pharmaceutical composition comprises more than about 0.5 ppm, more than about 1 ppm, more than about 10 ppm, more than about 50 ppm, more than about 100 ppm, more than about 500 ppm, more than about 1000 ppm, more than about 2000 ppm, more than about 3000 ppm, more than about 4000 ppm, less than about 5000 ppm, less than about 4000 ppm, less than about 3000 ppm, less than about 2000 ppm, less than about 1000 ppm, less than about 900 ppm, less than about 800 ppm, less than about 700 ppm, less than about 600 ppm, less than about 500 ppm, less than about 400 ppm, less than about 300 ppm, less than about 200 ppm, less than about 100 ppm, from about 0.5 ppm to about 5000 ppm, from about 1 ppm to about 5000 ppm, from about 5 ppm to about 5000 ppm, from about 10 ppm to about 5000 ppm, from about 10 ppm to about 4000 ppm, from about 10 ppm to about 3000 ppm, from about 10 ppm to about 2000 ppm, from about 10 ppm to about 1000 ppm, from about 10 ppm to about 500 ppm, from about 10 ppm to about 400 ppm, from about 10 ppm to about 300 ppm, from about 50 ppm to about 300 ppm, of Compound 2, or a pharmaceutically acceptable salt or solvate thereof.

In one embodiment, the pharmaceutical composition comprises from about 0.00005 weight % to about 0.5 weight % of Compound 2 or a pharmaceutically acceptable salt or solvate thereof. In one embodiment, the pharmaceutical composition comprises more than about 0.00005 weight %, more than about 0.0005 weight %, more than about 0.005 weight %, more than about 0.05 weight %, more than about 0.5 weight %, less than about 1.0 weight %, less than about 0.9 weight %, less than about 0.8 weight %, less than about 0.7 weight %, less than about 0.6 weight %, less than about 0.5 weight %, from about 0.00005 weight % to about 0.5 weight %, from about 0.0005 weight % to about 0.5 weight %, from about 0.005 weight % to about 0.5 weight %, from about 0.05 weight % to about 0.5 weight % of Compound 2 or a pharmaceutically acceptable salt or solvate thereof.

### Dosage Form

In some embodiments, the pharmaceutical compositions described herein are provided in unit dosage form. As used herein, a “unit dosage form” is a composition containing an amount of Compound 1 that is suitable for administration to an animal, preferably mammal, subject in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical compositions described herein are formulated as oral dosage forms. Suitable oral dosage forms include, for example, tablets, pills, sachets, or capsules. In some embodiments, the pharmaceutical composition comprises one or more additional pharmaceutically acceptable excipients. See, e.g., Remington: The Science and Practice of Pharmacy (Gennaro, 21s^{t} Ed. Mack Pub. Co., Easton, Pa. (2005) for a list of pharmaceutically acceptable excipients.

### Capsule

In some embodiments, the pharmaceutical composition is formulated as a capsule. In some embodiments, the pharmaceutical composition is formulated as a hard gel capsule. In some embodiments, the pharmaceutical composition is formulated as a soft gel capsule.

In some embodiments, the capsule is formed using materials which include, but are not limited to, natural or synthetic gelatin, pectin, casein, collagen, protein, modified starch, polyvinylpyrrolidone, acrylic polymers, cellulose derivatives, or any combinations thereof. In some embodiments, the capsule is formed using preservatives, coloring and opacifying agents, flavorings and sweeteners, sugars, gastroresistant substances, or any combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods and swallowed without chewing. In some embodiments, the shape and size of the capsule also vary. Examples of capsule shapes include, but are not limited to, round, oval, tubular, oblong, twist off, or a non-standard shape. The size of the capsule may vary according to the volume of the particulates. In some embodiments, the size of the capsule is adjusted based on the volume of the particulates and powders. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, the pharmaceutical composition described herein (e.g., capsule) is swallowed as a whole.

In some embodiments, the capsule comprises one or more pharmaceutically acceptable excipients. In some embodiments, the capsule is free of additional excipients.

In some embodiments, a capsule is developed, manufactured and commercialized for a drug substance that is insoluble. In some embodiments, a drug substance is insoluble if solubility is less than 0.002 mg/mL in water. In some embodiments, the capsule has a dose strength of up to 200 mg. In some embodiments, drug substance in the capsule is immediately released in a dissolution medium using USP apparatus I. In some embodiments, drug substance in the capsule is immediately released in a dissolution medium using USP apparatus II.

### Tablet

Insoluble drugs may be difficult to formulate using standard technologies such as high shear wet granulation. Optimum delivery of insoluble drugs may require complex technologies such as solid solutions amorphous dispersions (hot melt extrusion or spray drying), nano-formulations or lipid-based formulations. Hydrophobic drug substances may be considered insoluble according to USP criteria and may be known to be difficult to granulate with water and other excipients. This is likely due to most known excipients for immediate release formulations being water soluble or water-swellable. Making a tablet of a high dose drug substance that is insoluble may require a high concentration of the drug substance. However, as the drug concentration is increased above a certain level, formation of granules may become more and more difficult. Furthermore, at a certain drug load, it may become impossible.

In some embodiments, the pharmaceutical composition is formulated as a tablet.

In some embodiments, the tablet is made by compression, molding, or extrusion, optionally with one or more pharmaceutically acceptable excipient. In some embodiments, compressed tablets are prepared by compressing a compound of Formula I, II, III, and IV, or Compounds 1 or 2 in a free-flowing form, optionally mixed with pharmaceutically acceptable excipients. In some embodiments, molded tablets are made by molding a mixture of the powdered a compound of Formula I, II, III, and IV, or Compounds 1 or 2 moistened with an inert liquid diluent. In some embodiments, the tablet is prepared by hot-melt extrusion. In some embodiments, extruded tablets are made by forcing a mixture comprising a compound of Formula I, II, III, and IV, or Compounds 1 or 2 through an orifice or die under controlled conditions. In some embodiments, the tablet is coated or scored. In some embodiments, the tablet is formulated so as to provide slow or controlled release of a compound of Formula I, II, III, and IV, or Compounds 1 or 2. In some embodiments, a tablet is developed, manufactured and commercialized for a drug substance that is insoluble. In some embodiments, a drug substance is insoluble if solubility is less than 0.002 mg/mL in water. In some embodiments, the tablet has a dose strength of up to 200 mg. In some embodiments, drug substance in the tablet is immediately released in a dissolution medium using USP apparatus I. In some embodiments, drug substance in the tablet is immediately released in a dissolution medium using USP apparatus II.

In some embodiments, the tablet size is less than about 1000 mg, less than about 800 mg, less than about 600 mg, less than about 400 mg or less than about 200 mg. In some embodiments, the tablet has a dose strength of more than about 50 mg, more than about 100 mg, more than about 150 mg, more than about 200 mg, or more than about 250 mg. In some embodiments, the tablet size is less than about 1000 mg for a dose strength of more than about 50 mg. In some embodiments, the tablet size is less than 800 mg for a dose strength of more than about 100 mg. In some embodiments, the tablet size is less than 600 mg for a dose strength of more than about 150 mg. In some embodiment, the tablet size is less than 400 mg for a dose strength of more than about 200 mg. In some embodiments, the tablet size is less than 400 mg for a dose strength of 200 mg.

In some embodiments, more than about 20% of the tablet is dissolved in conventional dissolution media. In some embodiments, more than about 40% of the tablet is dissolved in conventional dissolution media. In some embodiments, more than about 50% of the tablet is dissolved in conventional dissolution media. In some embodiments, more than about 60% of the tablet is dissolved in conventional dissolution media. In some embodiments, more than about 70% of the tablet is dissolved in conventional dissolution media. In some embodiments, more than about 80% of the tablet is dissolved in conventional dissolution media. In some embodiments, more than about 20% of the tablet is dissolved in less than 24 hours in conventional dissolution media. In some embodiments, more than about 20% of the tablet is dissolved in less than 12 hours in conventional dissolution media. In some embodiments, more than about 20% of the tablet is dissolved in less than 6 hours in conventional dissolution media. In some embodiments, more than about 20% of the tablet is dissolved in less than 3 hours in conventional dissolution media. In some embodiments, more than about 20% of the tablet is dissolved in less than 2 hours in conventional dissolution media. In some embodiments, more than about 20% of the tablet is dissolved in less than 60 minutes in conventional dissolution media. In some embodiments, more than about 40% of the tablet is dissolved in less than 60 minutes in conventional dissolution media. In some embodiments, more than about 50% of the tablet is dissolved in less than 60 minutes in conventional dissolution media. In some embodiments, more than about 60% of the tablet is dissolved in less than 60 minutes in conventional dissolution media. In some embodiments, more than about 70% of the tablet is dissolved in less than 60 minutes in conventional dissolution media. In some embodiments, more than about 80% of the tablet is dissolved in less than 60 minutes in conventional dissolution media. In some embodiments, more than 70% of the tablet is dissolved in 60 minutes in conventional dissolution media.

In some embodiments, the tablet is produced at a commercial scale.

In some embodiments, the tablet comprises one or more pharmaceutically acceptable excipients.

In some embodiments, the tablet is coated with a coating material, e.g., a sealant. In some embodiments, the coating material is water soluble. In some embodiments, the coating material comprises a polymer, plasticizer, a pigment, or any combination thereof. In some embodiments, the coating material is in the form of a film coating, e.g., a glossy film, a pH independent film coating, an aqueous film coating, a dry powder film coating (e.g., complete dry powder film coating), or any combination thereof. In some embodiments, the coating material is highly adhesive. In some embodiments, the coating material provides low level of water permeation. In some embodiments, the coating material provides oxygen barrier protection. In some embodiments, the coating material allows immediate disintegration for fast release of Compound 1. In some embodiments, the coating material is pigmented, clear, or white. In some embodiments, the coating is an enteric coating. Exemplary coating materials include, without limitation, polyvinylpyrrolidone, polyvinyl alcohol, an acrylate-methacrylic acid copolymer, a methacrylate-methacrylic acid copolymer, cellulose acetate phthalate, cellulose acetate succinate, hydroxypropyl methylcellulose phthalate, hydroxypropyl methylcellulose acetate succinate, polyvinyl acetate phthalate, shellac, cellulose acetate trimellitate, sodium alginate, zein, and any combinations thereof.

### Oral Dosage Forms

In some embodiments, the pharmaceutical composition is formulated as an oral dosage form. In some embodiments, the oral dosage form is selected from the group consisting of a tablet, a capsule, a buccal tablet, a sub-lingual table, an orally-disintegrating tablet, a thin film, a liquid solution, a liquid suspension, a syrup, a powder, and solid crystals. In some embodiment, the oral dosage form is selected from the group consisting of a tablet, a capsule, a buccal tablet, a sub-lingual table, an orally-disintegrating tablet, a thin film, a liquid solution, a liquid suspension, a syrup, a powder, solid crystals, minitabs, coated pellets and sachets.

Oral dosage forms may include capsules, tablets, pills, powders or granules. Such forms may include forms that dissolve or disintegrate quickly in the oral environment. Such forms may be prepared with coatings and shells. In some embodiments, these oral dosage forms are capable of controlled or sustained release.

### Pharmaceutically Acceptable Excipients

In some embodiments, the pharmaceutical composition comprises a pharmaceutically acceptable excipient. In some embodiments, the composition is free of pharmaceutically acceptable excipients. The term “pharmaceutically acceptable excipient”, as used herein, means one or more compatible solid or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. In some embodiments, the pharmaceutically acceptable excipient is of sufficiently high purity and sufficiently low toxicity to render them suitable for administration preferably to an animal, preferably mammal, being treated.

Some examples of substances, which can serve as pharmaceutically acceptable excipients include:
Amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine.
Monosaccharides such as glucose (dextrose), arabinose, mannitol, fructose (levulose), and galactose.
Cellulose and its derivatives such as sodium carboxymethyl cellulose, ethyl cellulose, and methyl cellulose.
Solid lubricants such as talc, stearic acid, magnesium stearate and sodium stearyl fumarate.
Polyols such as propylene glycol, glycerin, sorbitol, mannitol, and polyethylene glycol.
Emulsifiers such as the polysorbates.
Wetting agents such as sodium lauryl sulfate, Tween , Span , alkyl sulphates, and alkyl ethoxylate sulphates.
Cationic surfactants such as cetrimide, benzalkonium chloride, and cetylpyridinium chloride.
Diluents such as calcium carbonate, microcrystalline cellulose, calcium phosphate, starch, pregelatinized starch, sodium carbonate, mannitol, and lactose.
Binders such as starches (corn starch and potato starch), gelatin, sucrose hydroxypropyl cellulose (HPC), polyvinylpyrrolidone (PVP), and hydroxypropyl methyl cellulose (HPMC).
Disintegrants such as starch, and alginic acid.
Super-disintegrants such as ac-di-sol, croscarmellose sodium, sodium starch glycolate and crospovidone.
Glidants such as silicon dioxide.
Coloring agents such as the FD&C dyes.
Sweeteners and flavoring agents, such as aspartame, saccharin, menthol, peppermint, and fruit flavors.
Preservatives such as benzalkonium chloride, PHMB, chlorobutanol, thimerosal, phenylmercuric, acetate, phenylmercuric nitrate, parabens, and sodium benzoate.
Tonicity adjustors such as sodium chloride, potassium chloride, mannitol, and glycerin.
Antioxidants such as sodium bisulfite, acetone sodium bisulfite, sodium formaldehyde, sulfoxylate, thiourea, and EDTA.
pH adjuster such as NaOH, sodium carbonate, sodium acetate, HCl, and citric acid.
Cryoprotectants such as sodium or potassium phosphates, citric acid, tartaric acid, gelatin, and carbohydrates such as dextrose, mannitol, and dextran.
Surfactants such as sodium lauryl sulfate. For example, cationic surfactants such as cetrimide (including tetradecyl trimethyl ammonium bromide with dodecyl and hexadecyl compounds), benzalkonium chloride, and cetylpyridinium chloride. Some examples of anionic surfactants are alkylsulphates, alkylethoxylate sulphates, soaps, carxylate ions, sulfate ions, and sulfonate ions. Some examples of non-ionic surfactants are polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols.
Organic materials such as carbohydrates, modified carbohydrates, lactose (including a-lactose, monohydrate spray dried lactose or anhydrous lactose), starch, pregelatinized starch, sucrose, mannitol, sorbital, cellulose (including powdered cellulose and microcrystalline cellulose).
Inorganic materials such as calcium phosphates (including anhydrous dibasic calcium hosphate, dibasic calcium phosphate or tribasic calcium phosphate).
Co-processed diluents.
Compression aids.
Anti-tacking agents such as silicon dioxide and talc.

### Amounts

In some embodiments, the pharmaceutical composition, in the form of a tablet or capsule, comprises between about 1 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 100 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 70 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 60 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 50 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 40 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 30 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 20 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 10 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 1 mg and about 5 mg of a compound of Formula I, II, III, and IV, or Compounds 1 or 2, or a pharmaceutically acceptable salt or solvate thereof. In some embodiments, the pharmaceutical composition, in the form of a tablet or capsule comprises about 1 mg of a compound of Formula I, II, III, and IV, or Compounds 1 or 2, or a pharmaceutically acceptable salt or solvate thereof. In some embodiments, the pharmaceutical composition, in the form of a tablet or capsule comprises about 5 mg of a compound of Formula I, II, III, and IV, or Compounds 1 or 2, or a pharmaceutically acceptable salt or solvate thereof.

In some embodiments, the pharmaceutical composition, in the form of a tablet or capsule, comprises between about 1 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 5 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 100 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 70 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 10 mg and about 60 mg of a compound of Formula I, II, III, and IV, or Compounds 1 or 2, or a pharmaceutically acceptable salt or solvate thereof.

In some embodiments, the pharmaceutical composition, in the form of a tablet or capsule, comprises between about 20 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 100 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 70 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 20 mg and about 60 mg of a compound of Formula I, II, III, and IV, or Compounds 1 or 2, or a pharmaceutically acceptable salt or solvate thereof.

In some embodiments, the pharmaceutical composition, in the form of a tablet or capsule, comprises between about 30 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 100 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 70 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 30 mg and about 60 mg of a compound of Formula I, II, III, and IV, or Compounds 1 or 2, or a pharmaceutically acceptable salt or solvate thereof.

In some embodiments, the pharmaceutical composition, in the form of a tablet or capsule, comprises between about 40 mg and about 500 mg of a compound of Formula I, II, III, and IV, or Compounds 1 or 2 , or a pharmaceutically acceptable salt or solvate thereof. In some embodiments, the pharmaceutical composition, in the form of a tablet or capsule, comprises between about 40 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 100 mg of a compound of Formula I, II, III, and IV, or Compounds 1 or 2, or a pharmaceutically acceptable salt or solvate thereof. In some embodiments, the

Pharmaceutical composition, in the form of a tablet or capsule, comprises between about 40 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 70 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 40 mg and about 60 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises about 50 mg of a compound of Formula I, II, III, and IV, or Compounds 1 or 2 , or a pharmaceutically acceptable salt or solvate thereof.

In some embodiments, the pharmaceutical composition, in the form of a tablet or capsule, comprises between about 50 mg and about 500 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 400 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 300 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 200 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 100 mg of A compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 90 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 80 mg of a compound of Formula I, II, III, and IV, or Compounds 1 or 2, or a pharmaceutically acceptable salt or solvate thereof. In some embodiments, the pharmaceutical composition, in the form of a tablet or capsule, comprises between about 50 mg and about 70 mg of a compound of Formula I, II, III, and IV, or Compounds 1 or 2, or a pharmaceutically acceptable salt or solvate thereof.

In some embodiments, the pharmaceutical composition comprises between about 100 mg and about 500 mg of a compound of Formula I, II, III, and IV, or Compounds 1 or 2, or a pharmaceutically acceptable salt or solvate thereof. In some embodiments, the pharmaceutical composition comprises between about 100 mg and about 400 mg of a compound of Formula I, II, III, and IV, or Compounds 1 or 2, or a pharmaceutically acceptable salt or solvate thereof. In some embodiments, the pharmaceutical composition comprises between about 100 mg and about 300 mg of a compound of Formula I, II, III, and IV, or Compounds 1 or 2, or a pharmaceutically acceptable salt or solvate thereof. In some embodiments, the pharmaceutical composition comprises between about 150 mg and about 250 mg of a compound of Formula I, II, III, and IV, or Compounds 1 or 2, or a pharmaceutically acceptable salt or solvate thereof. In some embodiments, the pharmaceutical composition comprises between about 100 mg and about 200 mg of a compound of Formula I, II, III, and IV, or Compounds 1 or 2, or a pharmaceutically acceptable salt or solvate thereof.

In some embodiments, the pharmaceutical composition comprises about 500 mg of a compound of Formula I, II, III, and IV, or Compounds 1 or 2, or a pharmaceutically acceptable salt or solvate thereof. In some embodiments, the pharmaceutical composition comprises about 400 mg of a compound of Formula I, II, III, and IV, or Compounds 1 or 2, or a pharmaceutically acceptable salt or solvate thereof. In some embodiments, the pharmaceutical composition comprises about 300 mg of a compound of Formula I, II, III, and IV, or Compounds 1 or 2, or a pharmaceutically acceptable salt or solvate thereof. In some embodiments, the pharmaceutical composition comprises about 250 mg of a compound of Formula I, II, III, and IV, or Compounds 1 or 2, or a pharmaceutically acceptable salt or solvate thereof. In some embodiments, the pharmaceutical composition comprises about 200 mg of a compound of Formula I, II, III, and IV, or Compounds 1 or 2, or a pharmaceutically acceptable salt or solvate thereof. In some embodiments, the pharmaceutical composition comprises about 150 mg of a compound of Formula I, II, III, and IV, or Compounds 1 or 2, or a pharmaceutically acceptable salt or solvate thereof. In some embodiments, the pharmaceutical composition comprises about 100 mg of a compound of Formula I, II, III, and IV, or Compounds 1 or 2, or a pharmaceutically acceptable salt or solvate thereof. In some embodiments, the pharmaceutical composition comprises about 90 mg of a compound of Formula I, II, III, and IV, or Compounds 1 or 2, or a pharmaceutically acceptable salt or solvate thereof. In some embodiments, the pharmaceutical composition comprises about 80 mg of a compound of Formula I, II, III, and IV, or Compounds 1 or 2, or a pharmaceutically acceptable salt or solvate thereof. In some embodiments, the pharmaceutical composition comprises about 70 mg of a compound of Formula I, II, III, and IV, or Compounds 1 or 2, or a pharmaceutically acceptable salt or solvate thereof. In some embodiments, the pharmaceutical composition comprises about 60 mg of a compound of Formula I, II, III, and IV, or Compounds 1 or 2, or a pharmaceutically acceptable salt or solvate thereof. In some embodiments, the pharmaceutical composition comprises about 50 mg of a compound of Formula I, II, III, and IV, or Compounds 1 or 2, or a pharmaceutically acceptable salt or solvate thereof. In some embodiments, the pharmaceutical composition comprises about 40 mg of a compound of Formula I, II, III, and IV, or Compounds 1 or 2, or a pharmaceutically acceptable salt or solvate thereof. In some embodiments, the pharmaceutical composition comprises about 30 mg of a compound of Formula I, II, III, and IV, or Compounds 1 or 2, or a pharmaceutically acceptable salt or solvate thereof. In some embodiments, the pharmaceutical composition comprises about 20 mg of a compound of Formula I, II, III, and IV, or Compounds 1 or 2, or a pharmaceutically acceptable salt or solvate thereof. In some embodiments, the pharmaceutical composition comprises about 10 mg of a compound of Formula I, II, III, and IV, or Compounds 1 or 2, or a pharmaceutically acceptable salt or solvate thereof.

### Particle Size

In some embodiments, the pharmaceutical composition, in the form of a tablet or a capsule, comprises a compound of Formula I, II, III, and IV, or Compounds 1 or 2, or a pharmaceutically acceptable salt or solvate thereof, in the form of microparticles. In some embodiments, microparticles of a compound of Formula I, II, III, and IV, or Compounds 1 or 2 have an average size from about 1 μm to about 100 μm. In some embodiments, microparticles of a compound of Formula I, II, III, and IV, or Compounds 1 or 2 have an average size from about 1 μm to about 50 μm. In some embodiments, microparticles of a compound of Formula I, II, III, and IV, or Compounds 1 or 2 have an average size from about 1 μm to about 30 μm. In some embodiments, microparticles of a compound of Formula I, II, III, and IV, or Compounds 1 or 2 have an average size from about 1 μm to about 20 μm. In some embodiments, microparticles of a compound of Formula I, II, III, and IV, or Compounds 1 or 2 have an average size from about 5 μm to about 15 μm. In some embodiments, microparticles of a compound of Formula I, II, III, and IV, or Compounds 1 or 2 have an average size from about 1 μm to about 10 μm. In some embodiments, microparticles of a compound of Formula I, II, III, and IV, or Compounds 1 or 2 have an average size from about 3 μm to about 10 μm. In some embodiments, microparticles of a compound of Formula I, II, III, and IV, or Compounds 1 or 2 have an average size from about 4 μm to about 9 μm.

In some embodiments, microparticles of a compound of Formula I, II, III, and IV, or Compounds 1 or 2 have an average size less than about 100 μm. In some embodiments, microparticles of a compound of Formula I, II, III, and IV, or Compounds 1 or 2 have an average size less than about 80 μm. In some embodiments, microparticles of a compound of Formula I, II, III, and IV, or Compounds 1 or 2 have an average size less than about 60 μm. In some embodiments, microparticles of a compound of Formula I, II, III, and IV, or Compounds 1 or 2 have an average size less than about 50 μm. In some embodiments, microparticles of a compound of Formula I, II, III, and IV, or Compounds 1 or 2 have an average size less than about 40 μm. In some embodiments, microparticles of a compound of Formula I, II, III, and IV, or Compounds 1 or 2 have an average size less than about 30 μm. In some embodiments, microparticles of a compound of Formula I, II, III, and IV, or Compounds 1 or 2 have an average size less than about 20 μm. In some embodiments, microparticles of a compound of Formula I, II, III, and IV, or Compounds 1 or 2 have an average size less than about 10 μm.

Described herein is a pharmaceutical composition comprising a compound of Formula I, II, III, and IV, or Compounds 1 or 2:

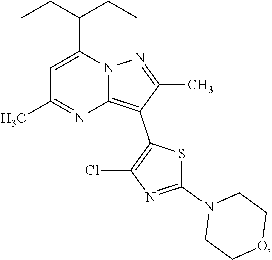 [SMILES: CCC(CC)c1cc(C)nc2c(-c3sc(N4CCOCC4)nc3Cl)c(C)nn12]

or a pharmaceutically acceptable salt or solvate thereof, which has a D₉₀ from about 1 μm to about 20 μm.

In some embodiments, the pharmaceutical composition comprising a compound of Formula I, II, III, and IV, or Compounds 1 or 2 , or a pharmaceutically acceptable salt or solvate thereof, has a D₉₀ more than about 1 μm, more than about 2 μm, more than about 3 μm, more than about 4 μ, more than about 5 μm, more than about 6 μm, more than about 7 μm, more than about 8 μm, more than about 9 μm, more than about 10 μm, more than about 11 μm, more than about 12 μm, more than about 13 μm, more than about 14 μm more than about 15 μm more than about 16 μm, more than about 17 μm, more than about 18 μm, more than about 19 μm, less than about 20 μm, less than about 19 μm, less than about 18 μm, less than about 17 μm, less than about 16 μm, less than about 15 μm, less than about 14 μm, less than about 13 μm, less than about 12 μm, less than about 11 μm, from about 1 μm to about 20 μm, from about 2 μm to about 19 pm, from about 3 μm to about 18 μm, from about 4 μm to about 17 μm, from about 5 μm to about 16 μm, from about 6 μm to about 15 μm, from about 7 μm to about 14 μm, from about 8 p.m to about 13 μm, from about 9 μm to about 13 μm, or from about 8 μm to about 12 μm.

### Pharmacokinetics

In some embodiments, a compound of Formula I, II, III, and IV, or Compounds 1 or 2 is formulated as a capsule or a tablet as to provide a Tmax of about 1 to about 8 hours in a subject. In some embodiments, a compound of Formula I, II, III, and IV, or Compounds 1 or 2 is formulated as a capsule or a tablet as to provide a Tmax of about 2 to about 7 hours in a subject. In some embodiments, a compound of Formula I, II, III, and IV, or Compounds 1 or 2 is formulated as a capsule or a tablet as to provide a Tmax of about 2 to about 6 hours in a subject. In some embodiments, a compound of Formula I, II, III, and IV, or Compounds 1 or 2 is formulated as a capsule or a tablet as to provide a Tmax of about 3 to about 5 hours in a subject.

In some embodiments, a compound of Formula I, II, III, and IV, or Compounds 1 or 2 is formulated as a capsule or a tablet as to provide a Tmax of about 8 hours in a subject. In some embodiments, a compound of Formula I, II, III, and IV, or Compounds 1 or 2 is formulated as a capsule or a tablet as to provide a Tmax of about 7 hours in a subject. In some embodiments, a compound of Formula I, II, III, and IV, or Compounds 1 or 2 is formulated as a capsule or a tablet as to provide a Tmax of about 6 hours in a subject. In some embodiments, a compound of Formula I, II, III, and IV, or Compounds 1 or 2 is formulated as a capsule or a tablet as to provide a Tmax of about 5 hours in a subject. In some embodiments, a compound of Formula I, II, III, and IV, or Compounds 1 or 2 is formulated as a capsule or a tablet as to provide a Tmax of about 4 hours in a subject. In some embodiments, a compound of Formula I, II, III, and IV, or Compounds 1 or 2 is formulated as a capsule or a tablet as to provide a Tmax of about 3 hours in a subject. In some embodiments, a compound of Formula I, II, III, and IV, or Compounds 1 or 2 is formulated as a capsule or a tablet as to provide a Tmax of about 2 hours in a subject. In some embodiments, a compound of Formula I, II, III, and IV, or Compounds 1 or 2 is formulated as a capsule or a tablet as to provide a Tmax of about 1 hour in a subject.

### Stability

The pharmaceutical compositions described herein are stable in various storage conditions including refrigerated, ambient and accelerated conditions. Stable as used herein refers to pharmaceutical compositions having about 95% or greater of the initial a compound of Formula I, II, III, and IV, or Compounds 1 or 2 amount and about 5% w/w or less total impurities or related substances at the end of a given storage period. The percentage of impurities is calculated from the amount of impurities relative to the amount of a compound of Formula I, II, III, and IV, or Compounds 1 or 2. Stability is assessed by HPLC or any other known testing method. In some embodiments, the stable pharmaceutical compositions have about 5% w/w, about 4% w/w, about 3% w/w, about 2.5% w/w, about 2% w/w, about 1.5% w/w, about 1% w/w, or about 0.5% w/w total impurities or related substances. In other embodiments, the stable pharmaceutical compositions have about 5% w/w total impurities or related substances. In yet other embodiments, the stable pharmaceutical compositions have about 4% w/w total impurities or related substances. In yet other embodiments, the stable pharmaceutical compositions have about 3% w/w total impurities or related substances. In yet other embodiments, the stable pharmaceutical compositions have about 2% w/w total impurities or related substances. In yet other embodiments, the stable pharmaceutical compositions have about 1% w/w total impurities or related substances.

At refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, refrigerated condition is 5±5° C. In some embodiments, refrigerated condition is about 0° C., about 0.1° C., about 0.2° C., about 0.3° C., about 0.4° C., about 0.5° C., about 0.6° C., about 0.7° C., about 0.8° C., about 0.9° C., about 1° C., about 1.1° C., about 1.2° C., about 1.3° C., about 1.4° C., about 1.5° C., about 1.6° C., about 1.7° C., about 1.8° C., about 1.9° C., about 2° C., about 2.1° C., about 2.2° C., about 2.3° C., about 2.4° C., about 2.5° C., about 2.6° C., about 2.7° C., about 2.8° C., about 2.9° C., about 3° C., about 3.1° C., about 3.2° C., about 3.3° C., about 3.4° C., about 3.5° C., about 3.6° C., about 3.7° C., about 3.8° C., about 3.9° C., about 4° C., about 4.1° C., about 4.2° C., about 4.3° C., about 4.4° C., about 4.5° C., about 4.6° C., about 4.7° C., about 4.8° C., about 4.9° C., about 5° C., about 5.1° C., about 5.2° C., about 5.3° C., about 5.4° C., about 5.5° C., about 5.6° C., about 5.7° C., about 5.8° C., about 5.9° C., about 6° C., about 6.1° C., about 6.2° C., about 6.3° C., about 6.4° C., about 6.5° C., about 6.6° C., about 6.7° C., about 6.8° C., about 6.9° C., about 7° C., about 7.1° C., about 7.2° C., about 7.3° C., about 7.4° C., about 7.5° C., about 7.6° C., about 7.7° C., about 7.8° C., about 7.9° C., about 8° C., about 8.1° C., about 8.2° C., about 8.3° C., about 8.4° C., about 8.5° C., about 8.6° C., about 8.7° C., about 8.8° C., about 8.9° C., about 9° C., about 9.1° C., about 9.2° C., about 9.3° C., about 9.4° C., about 9.5° C., about 9.6° C., about 9.7° C., about 9.8° C., about 9.9° C., or about 10° C. At accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, or at least 24 months. Accelerated conditions for the pharmaceutical compositions described herein include temperatures that are at or above ambient levels (e.g. 25±5° C.). In some instances, an accelerated condition is at about 40±2° C. In some instances, an accelerated condition is at about 35° C., about 40° C., about 45° C., about 50° C., about 55° C., or about 60° C. Accelerated conditions for the pharmaceutical compositions described herein also include relative humidity (RH) that are at or above ambient levels (55±10% RH). In other instances, an accelerated condition is above about 65% RH, about 70% RH, about 75% RH, or about 80% RH. In further instances, an accelerated condition is about 40 ° C. or 60° C. at ambient humidity. In yet further instances, an accelerated condition is about 40±2 ° C. at 75±5% RH humidity.

In some embodiments, the pharmaceutical compositions are stable at about 5±5° C. to about 25±5° C. for at least 12 months. In one embodiment, the pharmaceutical compositions are stable at about 5±5° C. for at least 12 months. In one embodiment, the pharmaceutical compositions are stable at about 25±5° C. for at least 12 months. In one embodiment, the pharmaceutical compositions are stable at about 5±5° C. for at least 24 months. In one embodiment, the pharmaceutical compositions are stable at about 25±5° C. for at least 24 months.

### Methods of Use

Disclosed herein is a method of treating congenital adrenal hyperplasia (CAH) in a subject in need thereof, comprising administering a pharmaceutical composition comprising a compound of Formula I, II, III, and IV, or Compounds 1 or 2, or a pharmaceutically acceptable salt or solvate thereof. In some embodiments, CAH is classic CAH. In some embodiments, CAH is non-classic CAH. In some embodiments, the methods described herein result in the reduction of a hormone level. Such hormones include deoxycorticosterone, 11-deoxycortisol, cortisol, corticosterone, aldosterone, pregnenolone, 17α-hydroxy pregnenolone, progesterone, 17α-hydroxy progesterone (17-OHP), dehydroepiandrosterone, androstenediol, androstenedione, testosterone, dihydrotestosterone, estrone, estradiol, estriol, and adrenocorticotropic hormone (ACTH). In some embodiments, the methods described herein result in the reduction of 17α-hydroxy progesterone (17-OHP). In some embodiments, the methods described herein result in the reduction of adrenocorticotropic hormone (ACTH), also known as corticotropin.

Also disclosed herein is a method of treating congenital adrenal hyperplasia (CAH) in a subject in need thereof, the method comprising:
(i) measuring a hormone level in the subject in need thereof;
(ii) administering a compound of Formula I, II, III and IV, or Compounds 1 or 2 or a pharmaceutically acceptable salt or solvate thereof;
(iii) repeating steps (i) and (ii) until the hormone level reaches a pre-determined range followed by a maintenance therapy of a daily dosing of a compound of Formula I, II, III, and IV, or Compounds 1 or 2.

In some embodiment, the hormone is 17α-Hydroxyprogesterone (17-OHP), adrenocorticotropic hormone (ACTH), testosterone, or androstenedione.

In some embodiment, the hormone is 17-OHP, and the pre-determined range is from about 200 ng/dL to about 400 ng/dL. In some embodiment, the hormone is 17-OHP, and the pre-determined range is less than about 400 ng/dL, less than about 350 ng/dL, less than about 300 ng/dL, less than about 250 ng/dL, or less than about 200 ng/dL.

In some embodiment, the hormone is ACTH, and the pre-determined range is below about 100 pg/mL. In some embodiment, the hormone is ACTH, and the pre-determined range is below about 100 pg/mL, below about 90 pg/mL, or below about 80 pg/mL.

In some embodiment, the hormone is testosterone and the pre-determined range is from about 14 ng/dL to about 76 ng/dL. In some embodiment, the hormone is testosterone and the pre-determined range is less than about 76 ng/dL, less than about 70 ng/dL, less than about 65 ng/dL, less than about 60 ng/dL, less than about 55 ng/dL, less than about 50 ng/dL, less than about 45 ng/dL, less than about 40 ng/dL, less than about 35 ng/dL, less than about 30 ng/dL, less than about 25 ng/dL, less than about 20 ng/dL, or less than about 15 ng/dL.

In some embodiment, the hormone is androstenedione and the pre-determined range is from about 30 ng/dL to about 200 ng/dL in males. In some embodiment, the hormone is androstenedione and the pre-determined range is less than about 200 ng/dL, less than about 150 ng/dL, less than about 100 ng/dL, less than about 50 ng/dL, or less than about 30 ng/dL in males

In some embodiment, the hormone is androstenedione and the pre-determined range is from about 40 ng/dL to about 150 ng/dL in females. In some embodiment, the hormone is androstenedione and the pre-determined range is less about 150 ng/dL, less about 100 ng/dL, less about 50 ng/dL, or less about 40 ng/dL in females.

In some embodiments, the methods described herein include administration of the pharmaceutical composition comprising a compound of Formula I, II, III, and IV, or Compounds 1 or 2 , or a pharmaceutically acceptable salt or solvate thereof once a month, twice a month, three times a month, once a week, twice a week, three times a week, once every two days, once a day, twice a day, three times a day, or four times a day. In some embodiments, the methods described herein administer a compound of Formula I, II, III, and IV, or Compounds 1 or 2 , or a pharmaceutically acceptable salt or solvate thereof once a day. In some embodiments, the methods described herein administer a compound of Formula I, II, III, and IV, or Compounds 1 or 2 , or a pharmaceutically acceptable salt or solvate thereof twice a day.

In some embodiments, the methods described herein include administration of about 1 mg to about 2000 mg of a compound of Formula I, II, III, and IV, or Compounds 1 or 2, or a pharmaceutically acceptable salt or solvate thereof, per day. In some embodiments, about 100 mg to about 1600 mg of a compound of Formula I, II, III, and IV, or Compounds 1 or 2, or a pharmaceutically acceptable salt or solvate thereof, is administered per day. In some embodiments, about 200 mg to about 1600 mg of a compound of Formula I, II, III, and IV, or Compounds 1 or 2, or a pharmaceutically acceptable salt or solvate thereof, is administered per day. In some embodiments, about 200 mg to about 1200 mg of a compound of Formula I, II, III, and IV, or Compounds 1 or 2, or a pharmaceutically acceptable salt or solvate thereof, is administered per day. In some embodiments, about 200 mg to about 1000 mg of a compound of Formula I, II, III, and IV, or Compounds 1 or 2, or a pharmaceutically acceptable salt or solvate thereof, is administered per day. In some embodiments, about 200 mg to about 800 mg of a compound of Formula I, II, III, and IV, or Compounds 1 or 2, or a pharmaceutically acceptable salt or solvate thereof, is administered per day. In some embodiments, about 100 mg to about 800 mg of a compound of Formula I, II, III, and IV, or Compounds 1 or 2 , or a pharmaceutically acceptable salt or solvate thereof, is administered per day. In some embodiments, about 200 mg to about 800 mg of a compound of Formula I, II, III, and IV, or Compounds 1 or 2 , or a pharmaceutically acceptable salt or solvate thereof, is administered per day. In some embodiments, about 100 mg to about 600 mg of a compound of Formula I, II, III, and IV, or Compounds 1 or 2 , or a pharmaceutically acceptable salt or solvate thereof, is administered per day. In some embodiments, about 200 mg to about 600 mg of a compound of Formula I, II, III, and IV, or Compounds 1 or 2 , or a pharmaceutically acceptable salt or solvate thereof, is administered per day. In some embodiments, about 300 mg to about 600 mg of a compound of Formula I, II, III, and IV, or Compounds 1 or 2 , or a pharmaceutically acceptable salt or solvate thereof, is administered per day. In some embodiments, about 100 mg to about 400 mg of a compound of Formula I, II, III, and IV, or Compounds 1 or 2 , or a pharmaceutically acceptable salt or solvate thereof, is administered per day. In some embodiments, about 200 mg to about 400 mg of a compound of Formula I, II, III, and IV, or Compounds 1 or 2, or a pharmaceutically acceptable salt or solvate thereof, is administered per day. In some embodiments, about 300 mg to about 400 mg of a compound of Formula I, II, III, and IV, or Compounds 1 or 2, or a pharmaceutically acceptable salt or solvate thereof, is administered each day.

In some embodiments, less than about 2000 mg a compound of Formula I, II, III, and IV, or Compounds 1 or 2, or a pharmaceutically acceptable salt or solvate thereof, is administered per day. In some embodiments, less than about 1800 mg a compound of Formula I, II, III, and IV, or Compounds 1 or 2, or a pharmaceutically acceptable salt or solvate thereof, is administered per day. In some embodiments, less than about 1600 mg a compound of Formula I, II, III, and IV, or Compounds 1 or 2 , or a pharmaceutically acceptable salt or solvate thereof, is administered per day. In some embodiments, less than about 1400 mg a compound of Formula I, II, III, and IV, or Compounds 1 or 2 , or a pharmaceutically acceptable salt or solvate thereof, is administered per day. In some embodiments, less than about 1200 mg a compound of Formula I, II, III, and IV, or Compounds 1 or 2, or a pharmaceutically acceptable salt or solvate thereof, is administered per day. In some embodiments, less than about 1000 mg a compound of Formula I, II, III, and IV, or Compounds 1 or 2, or a pharmaceutically acceptable salt or solvate thereof, is administered per day. In some embodiments, less than about 800 mg a compound of Formula I, II, III, and IV, or Compounds 1 or 2 , or a pharmaceutically acceptable salt or solvate thereof, is administered per day. In some embodiments, less than about 600 mg a compound of Formula I, II, III, and IV, or Compounds 1 or 2, or a pharmaceutically acceptable salt or solvate thereof, is administered per day. In some embodiments, less than about 500 mg a compound of Formula I, II, III, and IV, or Compounds 1 or 2, or a pharmaceutically acceptable salt or solvate thereof, is administered per day. In some embodiments, less than about 400 mg a compound of Formula I, II, III, and IV, or Compounds 1 or 2, or a pharmaceutically acceptable salt or solvate thereof, is administered per day. In some embodiments, less than about 300 mg a compound of Formula I, II, III, and IV, or Compounds 1 or 2, or a pharmaceutically acceptable salt or solvate thereof, is administered per day. In some embodiments, less than about 200 mg a compound of Formula I, II, III, and IV, or Compounds 1 or 2, or a pharmaceutically acceptable salt or solvate thereof, is administered per day.

In some embodiments, the methods described herein include administration of the pharmaceutical compositions described herein wherein the subject is in the fed state. In some embodiments, the methods described herein include administration of the pharmaceutical compositions described herein wherein the subject is in the fasted state.

In some embodiments, the methods described herein include administration of the pharmaceutical compositions described herein at bedtime.

In some embodiments, the methods described herein include administration of the pharmaceutical compositions described herein less than about 4 hours before sleep. In some embodiments, the methods described herein include administration of the pharmaceutical compositions described herein less than about 3 hours before sleep. In some embodiments, the methods described herein include administration of the pharmaceutical compositions described herein less than about 2 hours before sleep. In some embodiments, the methods described herein include administration of the pharmaceutical compositions described herein less than about 1 hour before sleep. In some embodiments, the methods described herein include administration of the pharmaceutical compositions described herein less than about 30 mins before sleep.

In some embodiments, the methods described herein include administration of the pharmaceutical compositions described herein in the evening.

In some embodiments, the methods described herein include administration of the pharmaceutical compositions described herein at about 11 pm at night. In some embodiments, the methods described herein include administration of the pharmaceutical compositions described herein at about 10 pm at night. In some embodiments, the methods described herein include administration of the pharmaceutical compositions described herein at about 9 pm at night. In some embodiments, the methods described herein include administration of the pharmaceutical compositions described herein at about 8 pm at night.

In some embodiments, the methods described herein include administration of the pharmaceutical compositions described herein at or before the expected circadian release of adrenocorticotropic hormone (ACTH). In some embodiments, the methods described herein include administration of the pharmaceutical compositions described herein about 3-4 hours before the expected circadian release of adrenocorticotropic hormone (ACTH).

### Combination Therapy

Disclosed herein is a method of treating congenital adrenal hyperplasia (CAH) in a subject in need thereof, comprising administering a combination of a compound of Formula I, II, III, and IV, or Compounds 1 or 2 , or a pharmaceutically acceptable salt or solvate thereof; and a glucocorticoid. In some embodiments, the amount of glucocorticoid administered is reduced as compared to a method not comprising administering a compound of Formula I, II, III, and IV, or Compounds 1 or 2 , or a pharmaceutically acceptable salt or solvate thereof.

In some embodiments, the methods described herein reduce the amount of a glucocorticoid administered from a supraphysiologic amount to a physiologic amount.

In some embodiments, the methods described herein reduce the symptoms associated with high-dose glucocorticoid therapy. In some embodiments, the symptoms associated with high-dose glucocorticoid therapy are obesity, insulin resistance, metabolic abnormalities, hypertension, cardiovascular diseases, or osteoporosis.

In some embodiments, the amount of glucocorticoid administered is reduced by about 5%, about 10%, about 15%, about 20%, about 25%, about 30%, about 35%, about 40%, about 45%, about 50%, about 55%, about 60%, about 65%, about 70%, about 80, or about 90% as compared to a method not comprising administering a compound of Formula I, II, III, and IV, or Compounds 1 or 2 , or a pharmaceutically acceptable salt or solvate thereof. In some embodiments, the amount of glucocorticoid administered is reduced by about 5%, about 10%, about 15%, about 20%, about 25%, about 30%, about 35%, about 40%, about 45%, about 50%, about 55%, or about 60% as compared to a method not comprising administering a compound of Formula I, II, III, and IV, or Compounds 1 or 2 , or a pharmaceutically acceptable salt or solvate thereof.

In some embodiments, the amount of glucocorticoid administered is reduced by about 1% to about 90%, about 1% to about 60%, about 1% to about 30%, about 1% to about 10%, about 10% to about 50%, about 10% to about 40%, about 10% to about 30%, about 15% to about 25%, about 20% to about 30%, about 5% to about 25%, about 20% to about 50%, about 30% to about 60%, or about 40% to about 70% as compared to a method not comprising administering a compound of Formula I, II, III, and IV, or Compounds 1 or 2 , or a pharmaceutically acceptable salt or solvate thereof.

In some embodiments, the glucocorticoid is administered at a dose between about 0.1 mg/day and about 25 mg/day. In some embodiments, the glucocorticoid is administered at a dose between about 1 mg/day and about 20 mg/day. In some embodiments, the glucocorticoid is administered at a dose between about 1 mg/day and about 15 mg/day. In some embodiments, the glucocorticoid is administered at a dose between about 1 mg/day and about 12 mg/day. In some embodiments, the glucocorticoid is administered at a dose between about 1 mg/day and about 11 mg/day. In some embodiments, the glucocorticoid is administered at a dose between about 1 mg/day and about 10 mg/day. In some embodiments, the glucocorticoid is administered at a dose between about 1 mg/day and about 9 mg/day. In some embodiments, the glucocorticoid is administered at a dose between about 1 mg/day and about 8 mg/day. In some embodiments, the glucocorticoid is administered at a dose between about 1 mg/day and about 7 mg/day. In some embodiments, the glucocorticoid is administered at a dose between about 1 mg/day and about 6 mg/day. In some embodiments, the glucocorticoid is administered at a dose between about 1 mg/day and about 5 mg/day. In some embodiments, the glucocorticoid is administered at a dose between about 1 mg/day and about 4 mg/day. In some embodiments, the glucocorticoid is administered at a dose between about 1 mg/day and about 3 mg/day. In some embodiments, the glucocorticoid is administered at a dose between about 1 mg/day and about 2 mg/day. In some embodiments, the glucocorticoid is administered at a dose between about 3 mg/day and about 13 mg/day. In some embodiments, the glucocorticoid is administered at a dose between about 5 mg/day and about 11 mg/day. In some embodiments, the glucocorticoid is administered at a dose between about 8 mg/day and about 11 mg/day. In some embodiments, the glucocorticoid is administered at a dose between about 9 mg/day and about 12 mg/day. In some embodiments, the glucocorticoid is administered at a dose between about 9 mg/day and about 10 mg/day. In some embodiments, the glucocorticoid is administered at a dose between about 5 mg/day and about 10 mg/day.

In some embodiments, a compound of Formula I, II, III, and IV, or Compounds 1 or 2 , or a pharmaceutically acceptable salt or solvate thereof, and the glucocorticoid are administered concurrently. In some embodiments, a compound of Formula I, II, III, and IV, or Compounds 1 or 2 , or a pharmaceutically acceptable salt or solvate thereof, and the glucocorticoid are administered in one pharmaceutical composition. In some embodiments, a compound of Formula I, II, III, and IV, or Compounds 1 or 2 , or a pharmaceutically acceptable salt or solvate thereof, and the glucocorticoid are administered concurrently in separate pharmaceutical compositions.

In some embodiments, a compound of Formula I, II, III, and IV, or Compounds 1 or 2 , or a pharmaceutically acceptable salt or solvate thereof, and the glucocorticoid are administered sequentially. In some embodiments, a compound of Formula I, II, III, and IV, or Compounds 1 or 2, or a pharmaceutically acceptable salt or solvate thereof, and the glucocorticoid are administered within 24 hours. In some embodiments, a compound of Formula I, II, III, and IV, or Compounds 1 or 2, or a pharmaceutically acceptable salt or solvate thereof, and the glucocorticoid are administered within 12 hours. In some embodiments, a compound of Formula I, II, III, and IV, or Compounds 1 or 2, or a pharmaceutically acceptable salt or solvate thereof, and the glucocorticoid are administered within 8 hours. In some embodiments, a compound of Formula I, II, III, and IV, or Compounds 1 or 2 , or a pharmaceutically acceptable salt or solvate thereof, and the glucocorticoid are administered within 6 hours. In some embodiments, a compound of Formula I, II, III, and IV, or Compounds 1 or 2 , or a pharmaceutically acceptable salt or solvate thereof, and the glucocorticoid are administered within 4 hours. In some embodiments, a compound of Formula I, II, III, and IV, or Compounds 1 or 2 , or a pharmaceutically acceptable salt or solvate thereof, and the glucocorticoid are administered within 2 hours. In some embodiments, a compound of Formula I, II, III, and IV, or Compounds 1 or 2 , or a pharmaceutically acceptable salt or solvate thereof, and the glucocorticoid are administered within 1 hour. In some embodiments, a compound of Formula I, II, III, and IV, or Compounds 1 or 2, or a pharmaceutically acceptable salt or solvate thereof, and the glucocorticoid are administered within 30 minutes. In some embodiments, a compound of Formula I, II, III, and IV, or Compounds 1 or 2 , or a pharmaceutically acceptable salt or solvate thereof, and the glucocorticoid are administered within 10 minutes.

In some embodiments, the glucocorticoid is beclomethasone, betamethasone, budesonide, cortisone, dexamethasone, hydrocortisone, methylprednisolone, prednisolone, prednisone, or triamcinolone. In some embodiments, the glucocorticoid is hydrocortisone.

In some embodiments, the glucocorticoid is hydrocortisone and the dose administered is less than the recommended dose of 15-25 mg/day.

In some embodiments, the glucocorticoid is prednisone and the dose administered is less than the recommended dose of 5-7.5 mg/day.

In some embodiments, the glucocorticoid is prednisolone and the dose administered is less than the recommended dose of 4-6 mg/day.

In some embodiments, the glucocorticoid is dexamethasone and the dose administered is less than the recommended dose of 0.25-0.5 mg/day.

Disclosed herein is a method of treating congenital adrenal hyperplasia (CAH) in a subject in need thereof, comprising administering a combination of a compound of Formula I, II, III, and IV, or Compounds 1 or 2 , or a pharmaceutically acceptable salt or solvate thereof; a glucocorticoid; and optionally a mineralcorticoid. In some embodiments, the mineralocorticoid is fludrocortisone and the dose is less than the recommended dose of 0.05-0.2 mg/day.

### EXAMPLES

The following examples further illustrate the invention but should not be construed as in any way limiting its scope. In particular, the processing conditions are merely exemplary and can be readily varied by one of ordinary skill in the art.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Unless defined otherwise, technical and scientific terms used herein have the same meaning as is commonly understood by one of skill in the art to which this invention belongs.

### Example 1

Pharmaceutical Composition

The pharmaceutical composition is manufactured as size 1 white hard gelatin capsules containing 200 mg of Compound 1 micronized to and average size of 10 microns or less. The pharmaceutical composition contains no additional excipients.

### Example 2

Stability of the Pharmaceutical Composition

The pharmaceutical composition is a Compound 1 neat-filled into size 0 capsules with no added excipients, in 3 strength configurations: 1-mg, 5-mg, and 50-mg. The capsules were blister packaged in a polyvinyl chloride (PVC)-based film.

Under long term and accelerated conditions, no significant trend was observed in the three lots for any of the attributes evaluated throughout the course of the stability study.

The data demonstrated that the pharmaceutical composition is stable for a minimum of 6 months (end of study). No adverse trends were observed under long term and accelerated conditions. The assay results were consistent through the entire study and no new related substances species were observed during the stability study. The reported stability results for lots stored in a blister packaging configuration are considered supportive for the updated packaging configuration of a 30-mL HDPE bottle, induction seal, and a child resistant cap. There are no excipients in either configuration and both configurations provide protection from light.

### Example 3

Phase 1 Clinical Studies

Compound 1 has been investigated in 2 Phase 1 studies in healthy adult volunteers.

Study 1 was the first-in-human study that investigated the safety, tolerability, and PK of single-escalating doses of Compound 1, given orally, to healthy adult subjects. Safety and tolerability assessments were made over a wide range of single oral doses, and dose escalation did not proceed until safety data from the preceding doses had been reviewed. The data from this study were used for the selection of doses for Study 2.

The 2-part multiple-dose study, Study 2, determined the safety and tolerability of repeated daily doses of Compound 1 and investigated the effects on biomarkers of relevance for the treatment of alcohol dependence. Part B investigated the interaction of Compound 1 with midazolam (a cytochrome P450 3A4 [CYP3A4] substrate), determining whether Compound 1 significantly inhibited the metabolism of drugs that are metabolized by CYP3A4.

In Study 1, Compound 1 was administered to healthy adult subjects as a single PO dose of 2, 10, 50, 150, 400, or 800 mg in the fed state and 150 mg in the fasted state. Absorption occurred moderately late, achieving peak Cₘₐₓ between 4 to 6 hours following dosing in the fed state.

Table 5 provides a summary of the PK parameters at each dose level. When Compound 1 was given in the fed state, median time to reach maximum plasma concentration (Tₘₐₓ) occurred between 4 and 6 hours. The median Tₘₐₓ was 10.05 hours when Compound 1 was given in the fasted state at 150 mg and ranged between 6 and 12 hours, suggesting possible delayed absorption in the fasted state. Mean half-life (t_{1/2}) after a single PO dose (fed and fasted state) was between 31 and 44 hours, ranging from 11 to 101 hours. Apparent volume of distribution (V_{z}/F) was large and appeared highly variable with greatest variability observed at the 2 highest dose levels of 400 mg and 800 mg.

TABLE 1
Summary of Pharmacokinetic Parameters of Compound 1 Following
Single Oral Dose Administration in Healthy Volunteers (all Fed)

| | Geometric Mean (CV %) | | | | | |
|---|---|---|---|---|---|---|
| PK | Analyte = Plasma Compound 1 | | | | | |
| Parameters | 2 mg | 10 mg | 50 mg | 150 mg | 400 mg | 800 mg |
| N | 6 | 6 | 6 | 6 | 6 | 6 |
| Cₘₐₓ (ng/mL) | 0.867 | 3.01 | 19.5 | 93.4 | 207 | 382 |
| | (53) | (87) | (39) | (22) | (92) | (110) |
| Tₘₐₓ^{a} (h) | 6.00 | 4.00 | 4.00 | 4.00 | 4.00 | 6.00 |
| | (4.00- | (2.00- | (3.00- | (3.00- | (3.00- | (4.00- |
| | 6.00) | 6.00) | 6.00) | 6.00) | 6.00) | 6.00) |
| t_{1/2}b (h) | NC | NC | 31 | 29.2 | 44.2 | 41.9 |
| | (NC) | (NC) | (10.8- | (20.0- | (20.2- | (24.1- |
| | | | 53.4) | 41.5) | 101) | 67.8) |
| AUC₀₋ₜ_{ₗₐₛₜ} | NC | NC | 152 | 891 | 2300 | 4390 |
| (ng · h/mL) | (NC) | (NC) | (60) | (26) | (103) | (100) |
| AUC_{0-∞} | NC | NC | 165 | 956 | 2580 | 4850 |
| (ng · h/mL) | (NC) | (NC) | (64) | (25) | (93) | (95) |
| CL/F (L/h) | NC | NC | 302 | 157 | 155 | 165 |
| | (NC) | (NC) | (64) | (25) | (93) | (95) |
| V_{Z}/F (L) | NC | NC | 13500 | 6620 | 9890 | 9970 |
| | (NC) | (NC) | (45) | (38) | (212) | (144) |
| V_{SS}/F (L) | NC | NC | 8080 | 4600 | 6580 | 6820 |
| | (NC) | (NC) | (36) | (33) | (152) | (120) |

AUC = area under the plasma concentration-time curve;
CL/F = oral clearance;
Cₘₐₓ = maximum plasma concentration;
CV = coefficient of variation;
NC = not calculable;
t_{1/2} = elimination half-life;
Tₘₐₓ = time to reach maximum plasma concentration;
V_{SS}/F = volume of distribution at steady state;
V_{Z}/F = volume of distribution at terminal phase
^{a}Median (range)
bGeometric mean (range)

As part of this single-dose escalation study, a food effect PK investigation was performed to examine Compound 1 exposures in both the fed and fasted states at the 150-mg dose level. A total of 6 subjects were administered 150 mg Compound 1 in each of these 2 dosing groups. Of these 6 subjects, 4 subjects received Compound 1 at the same dose in both fed and fasted states. The administration of Compound 1 in the fasted state resulted in a much flatter mean concentration-time (i.e., with significantly lower absorption) profile when compared against the mean profile at the same dose, given within 5 minutes after a standardized breakfast meal. The mean AUC_{0-∞}, and Cₘₐₓ values for a 150-mg dose in the fed state was approximately 3- and 11-fold greater than that of the fasted state, respectively. Table 2 provides a summary of the PK parameters at the 150-mg dose level under both fed and fasted state conditions.

TABLE 2
Summary of Pharmacokinetic Parameters of Compound
1 Following Single Oral Dose Administration in
Healthy Volunteers - Fed vs. Fasted State

| | Geometric Mean (CV %) | | | |
|---|---|---|---|---|
| Pharmacokinetic | Analyte = Plasma Compound 1 | | | |
| Parameters | 150 mg Fed | | 150 mg Fasted | |
| N | 6 | | 6 | |
| Cₘₐₓ (ng/mL) | 93.4 | (22) | 8.22 | (83) |
| Tₘₐₓ^{a} (h) | 4.00 | (3.00-6.00) | 10.05 | (6.00-12.00) |
| t_{1/2}^{b} (h) | 29.2 | (20.0-41.5) | 38.4 | (24.2-88.5) |
| AUC₀₋ₜ_{ₗₐₛₜ} (ng · h/mL) | 891 | (26) | 288 | (48) |
| AUC_{0-∞} (ng · h/mL) | 956 | (25) | 331 | (44) |
| CL/F (L/h) | 157 | (25) | 454 | (44) |
| V_{Z}/F (L) | 6620 | (38) | 25100 | (81) |
| V_{SS}/F (L) | 4600 | (33) | 25200 | (68) |

AUC = area under the plasma concentration-time curve; CL/F = apparent total body clearance; Cₘₐₓ = maximum plasma concentration; CV = coefficient of variation; N = number of subjects; NC = not calculable; PK = pharmacokinetic; Tₘₐₓ = time to reach maximum plasma concentration; t½ = elimination half-life; VₛₛF = apparent volume of distribution at steady state during the terminal phase after extravascular administration; V_{z}/F = apparent volume of distribution during the terminal phase after extravascular administration.
^{a}Median (range)
^{b}Geometric mean (range)

PK parameters AUC_{0-∞} and Cₘₐₓ were analyzed separately for dose proportionality for Compound 1 from 50 to 800 mg when administered in the fed state. The analysis results suggested that for every doubling of dose, AUC_{0-∞} can be expected to increase 1.74 times more than what would be expected under dose proportionality. Cₘₐₓ appeared more than dose proportional but the formal test was inconclusive as the 90% confidence interval were partially within the 0.8-1.25 interval. Dose proportionality across administered doses in the fed state could not be concluded on the basis of AUC_{0-∞} or Cₘₐₓ.

PK were also evaluated in the multiple-dose, dose-escalation study, Study 2. In Part A of the study, subjects were divided into 3 cohorts and received 50, 150, or 200 mg Compound 1 or placebo for 14 consecutive days (at least 6 subjects received Compound 1 and 2 subjects received placebo in each cohort). Blood concentrations of Compound 1 were close to steady-state levels after 2 weeks of dosing and the accumulation ratio was between 2.51 to 3.65. Part B investigated the interaction of Compound 1 with midazolam (a CYP3A4 substrate), thereby determining whether this compound significantly inhibited the metabolism of drugs that are metabolized by CYP3A4 serial blood samples were collected to determine plasma concentrations of study drug after a single dose of Compound 1 had been administered and at steady state. All dosing occurred in the fed state. An assessment of diurnal cortisol levels, plus when under conditions of glucose clamp, were also carried out both prior to and during the dosing period.

Overall concentration time profiles of Compound 1 showed that absorption was moderately delayed with Cₘₐₓ achieved at a median of 5 hours following oral dosing. Consistent with the single-dose study (Study 1), concentrations appeared to decline in a bi-exponential manner, characterized by a rapid decrease within the first 24 hours. Following multiple daily dosing for 2 weeks, the t_{1/2} of Compound 1 exceeded 100 hours; therefore, an accumulation ratio of Compound 1 was between 2.51 to 3.65 (see Table 3). The Tₘₐₓ appeared to be consistent across doses. Overall half-lives, weight normalized CL/F and V/F were consistent for 150 and 200 mg. However, the values for these latter 2 parameters were almost doubled at the 50-mg dose level. Variability (CV%) for apparent clearance and volume of distribution were large and not reduced with weight-normalization.

TABLE 3
Summary of Noncompartmental Pharmacokinetic Parameters of
Compound 1 After Single (Day 1) and Multiple (Day 14) Oral Doses
of 50 mg, 150 mg, and 200 mg of Compound 1 in Part A of the Study

| | Geometric Mean | | | | | |
|---|---|---|---|---|---|---|
| | (CV %) | | | | | |
| | 50 mg | 150 mg | 200 mg | 50 mg | 150 mg | 200 mg |
| | Day 1 | Day 1 | Day 1 | Day 14 | Day 14 | Day 14 |
| N | 8 | 9 | 7 | 8 | 9 | 6 |
| Cₘₐₓ(ng/mL) | 22.7 | 127 | 143 | 50.3 | 222 | 314 |
| | (59) | (52) | (62) | (56) | (58) | (93) |
| Tₘₐₓ^{a} (hr) | 4.52 | 5.00 | 5.00 | 5.00 | 5.00 | 5.00 |
| | (3.00- | (5.00- | (2.00- | (3.00- | (3.00- | (3.00- |
| | 10.00) | 6.00) | 5.00) | 5.00) | 5.03) | 5.00) |
| Effective t_{1/2}^{b} | NC | NC | NC | 37.7 | 32.7 | 52.0 |
| (hr) | NC | NC | NC | (27.4) | (29.7) | |
| AUC_{0-∞} | NC | NC | NC | 980 | 4680 | 5660 |
| (ng · hr/mL) | NC | NC | NC | (53.8) | (104) | (122) |
| AUC₀₋₂₄ₕ | 97.6 | 590 | 559 | 273 | 1480 | 2040 |
| (ng · hr/mL) | (58) | (56) | (55^{c}) | (56) | (66) | (98) |
| C_{avg} (ng/mL) | NC | NC | NC | 11.4 | 61.7 | 85.0 |
| | NC | NC | NC | (56) | (66) | (98) |
| CLₐₐ/F (L/hr) | NC | NC | NC | 183 | 101 | 98 |
| | NC | NC | NC | (56) | (66) | (98) |

WT-norm

| | | | | | | |
|---|---|---|---|---|---|---|
| Clₐₐ/F | NC | NC | NC | 2.88 | 1.42 | 1.50 |
| (L/hr/kg) | NC | NC | NC | (46.9) | (72.9) | (89.9) |
| Vz/F (L) | NC | NC | NC | 33500 | 17600 | 16300 |
| | NC | NC | NC | (90) | (59) | (76) |
| Vss/F (L) | NC | NC | NC | 12900 | 6170 | 5080 |
| | NC | NC | NC | (91) | (40) | (78) |

WT-norm

| | | | | | | |
|---|---|---|---|---|---|---|
| Vss/F (L/kg) | NC | NC | NC | 204 | 86.5 | 77.4 |
| | NC | NC | NC | (71.3) | (41.0) | (68.6) |
| | NC | NC | NC | 2.80 | 2.51 | 3.65 |
| R_{A} | NC | NC | NC | (27.4) | (29.7) | (34.4) |

AUC = area under the plasma concentration-time curve;
CL/F = apparent clearance;
Cₘₐₓ = maximum plasma concentration;
R_{A} = accumulation ratio calculated as Day 14 AUC₀₋₂₄/Day 1 AUC₀₋₂₄;
t_{1/2} = terminal half-life;
effective t_{1/2} = half-life calculated by accumulation ratio;
Tₘₐₓ = time to maximum plasma concentration;
Vss/F = volume of distribution at steady state;
Vz/F = volume of distribution at terminal phase;
WT-norm = weight normalized
^{a}Median (range).
^{b}Geometric mean (range).
^{c}n = 6, Dropout Subject 306 not included in the calculation of summary statistics.

Table 8 presents the results of the dose proportionality assessment for the AUC₀₋₂₄ and Cₘₐₓ over the tested dose range. For AUC₀₋₂₄ and Cₘₐₓ, the adjusted mean slope at Day 1 and Day 14 were all above the value of 1, suggesting a slightly more than proportional increase of AUC₀₋₂₄ and Cₘₐₓ values with increasing doses.

TABLE 4
Summary of Assessment of Dose Proportionality as
Assessed by the Power Model for Plasma Compound 1

| | | Mean | Standard | 90% CI | |
|---|---|---|---|---|---|
| Parameter | Day | Slope | Error | for Slope | CV % |
| AUC₀₋₂₄ | 1 | 1.40 | 0.194 | (1.069, 1.737) | 59.3 |
| (ng hr/mL) | 14 | 1.48 | 0.221 | (1.104, 1.864) | 69.1 |
| Cₘₐₓ | 1 | 1.41 | 0.185 | (1.092, 1.728) | 57.5 |
| (ng/mL) | 14 | 1.33 | 0.210 | (0.972, 1.693) | 64.8 |

AUC = area under the plasma concentration-time curve;
Cₘₐₓ = maximum plasma concentration Safety

The safety of Compound 1 was evaluated in 2 Phase 1 studies in healthy adult volunteers (Study 1 and Study 2). In both studies, adverse events (AEs), clinical laboratory tests, vital signs (supine blood pressure and pulse rate), and electrocardiograms (ECGs) were evaluated. Overall, in Study 1, Compound 1 was well-tolerated. Compound 1, when administered as multiple doses up to 200 mg, was generally well tolerated in the healthy subject population studied.

In Study 1, the effects upon biomarkers of relevance for the treatment of alcohol dependence were investigated. Five clusters of an Addiction Research Center Inventory Questionnaire (ARCI-49) were used to compare the effect of Compound 1 vs. placebo: Morphine-Benzedrine Group Scale measuring euphoria; Lysergic-Acid-Diethylamide Group Scale estimating dysphoric and somatic changes; Pentobarbital-Chlorpromazine-Alcohol Group Scale measuring sedation; Benzedrine Group (BG) Scale measuring intellectual efficiency and energy; Amphetamine Group Scale measuring effects of d-amphetamine, respectively. No systematic pattern or dose-response for the change from baseline or for the difference over placebo in each cluster was observed.

In summary, single, oral doses up to 800 mg and multiple doses up to 200 mg of Compound 1 were well-tolerated by healthy male and female subjects.

### Example 4

Phase 2 Multiple-Dose, Dose-Escalation Clinical Studies

Cohort A of the Phase 2 Study includes a 6-week, multiple-dose, dose escalation study of Compound 1 for the treatment of adults with classic CAH. After screening, eligible patients will be enrolled into a 6-week treatment period followed by a 4-week washout/safety follow-up period.

This cohort will be conducted in approximately 9 patients, who will receive Compound 1 daily for up to 6 weeks. Compound 1 will be administered as an oral daily dose. Patients will undergo titration of Compound 1 through three escalating dosage strengths at 2-week intervals. Patients will have overnight PK/PD assessments performed at baseline, which include an pre-dose overnight assessment and a post-dose overnight assessment for PK/PD following administration of the first dose. At the end of each 2-week dosing period, patients will return for single overnight visits for steady-state PK/PD assessments. A follow-up outpatient visit will occur 30 days after their last dose. Upon completion of the initial cohort (Cohort A), the study will proceed to a multiple ascending dose (MAD) design with up to 3 sequential cohorts (Cohorts B, C, and D) to further evaluate the safety, PK, and PD of various SPROO1 dosing regimens and to identify an optimal dose regimen. Each cohort will undergo a 2-week run-in period, a 2-week treatment period, and a 30-day washout and safety follow-up period. During the run-in period, which will occur during screening, subjects will document in a paper diary each dose of glucocorticoid medication taken, the time of each meal, and the time they went to bed and woke up each day, to ensure compliance with background glucocorticoid regimens and the stability of their daily routine.

Patients in Cohort B will receive study drug at 200 mg BID, with a dose in the morning and a dose in the evening, either with a meal or consumption of a standardized snack. For Cohorts C and D, the dose level and the frequency and timing of dosing will be determined based on interim data from the previous cohorts. However, the dose level of each successive cohort will be capped at twice the daily dose level of the previous cohort.

### Study Design

Study Type: Interventional

Primary Purpose: Treatment

Study Phase: Phase 2

Interventional Study Model: Sequential Assignment

Number of Cohorts: Up to 4

Masking: No masking

Allocation: Non-Randomized

Enrollment: Up to approximately 27 [Anticipated]

### Arms and Interventions

| Arms | Assigned Interventions |
|---|---|
| Experimental: Cohort A | Drug: Compound 1 |
| The first cohort of 9 patients will | 200-mg capsules |
| be administered Compound 1 at dose | |
| strength of 200 mg daily for 2 weeks, | |
| and escalating through 600 mg per day | |
| for 2 weeks and 1,000 mg per day for | |
| 2 weeks. | |
| Experimental: Cohort B | Drug: Compound 1 |
| Cohort B patients will be administered | 200-mg capsules |
| Compound 1 at a dose strength of 200 | |
| mg twice daily (BID) for 2 weeks. | |
| Experimental: Cohort C | Drug: Compound 1 |
| The dose/dose regimen for Cohort C will | 50-mg or 200 mg capsules |
| be determined based on an interim review | |
| of safety and PK/PD data from the | |
| previous Cohorts | |
| Experimental: Cohort D | Drug: Compound 1 |
| The dose/dose regimen for Cohort C will | 50-mg or 200 mg capsules |
| be determined based on an interim review | |
| of safety and PK/PD data from the | |
| previous Cohorts | |

### Outcome Measures

### Primary Outcome Measure:

1. To evaluate the safety of Compound 1 in subjects with CAH.
2. To assess the efficacy of Compound 1 in subjects with classic CAH as measured by percent and absolute change in 17-OHP compared to baseline.

### Secondary Outcome Measure:

3. To explore the dose(s) of Compound 1 that cause pharmacodynamics changes in plasma concentrations of ACTH, androstenedione, and testosterone, as measured by the absolute and percent change from baseline by dose.
4. To determine pharmacokinetics of Compound 1 in subjects with CAH.
5. To explore potential relationships between pharmacodynamics and pharmacokinetics.

### Exploratory

7. To explore the dose(s) of Compound 1 that cause changes in pharmacodynamics biomarkers in urine, as measured by the absolute and percent change from baseline by dose.

### Eligibility

Minimum Age: 18 Years

Maximum Age:

Sex: All

Gender Based: No

Accepts Healthy Volunteers: No

Criteria: Inclusion

### Criteria:

### Inclusion Criteria:

Male and female patients age 18 or older.

Documented diagnosis of classic CAH due to 21-hydroxylase deficiency

Elevated 17-OHP at screening

On a stable glucocorticoid replacement regimen for a minimum of 30 days

### Exclusion Criteria:

Clinically significant unstable medical condition, illness, or chronic disease

Clinically significant psychiatric disorder.

Clinically significant abnormal laboratory finding or assessment

History of bilateral adrenalectomy or hypopituitarism

Pregnant or nursing females

Use of any other investigational drug within 30 days

Unable to understand and comply with the study procedures, understand the risks, and/or unwilling to provide written informed consent.

### Results:

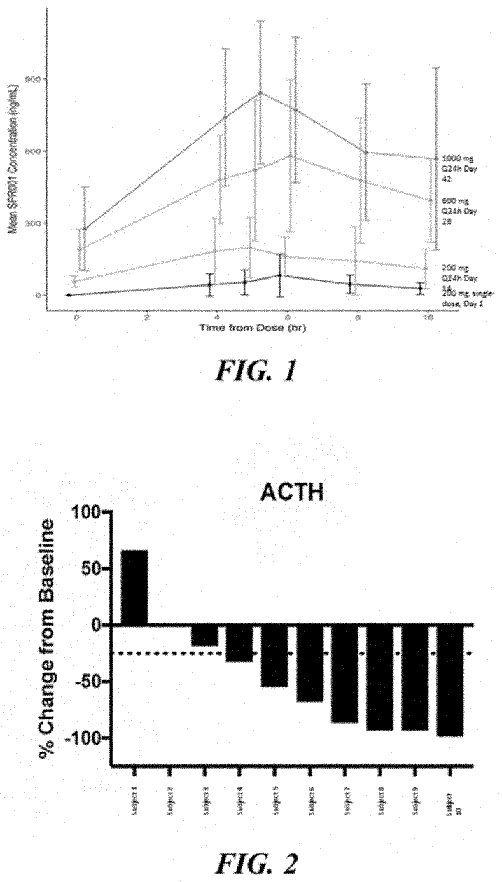

The phase 2 study showed that Compound 1 was generally well-tolerated. The study established a range of safe doses after exploring a wide range of doses (5-fold range) (see FIG. 1).

With respect to patient-level response to Compound 1, 80% showed reduced ACTH (see FIG. 2). Generally, attenuation of ACTH demonstrates target engagement and functional CRF₁ receptor antagonism. 80% of patient subjects demonstrated reduction in ACTH. 70% of subject demonstrated more than 25% reduction in ACTH. 40% of subjects were in the normal range after treatment.

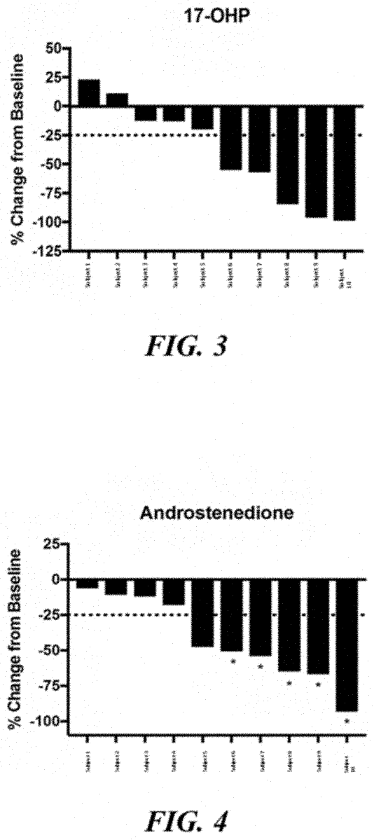

A reduction of 17-OHP demonstrates “control” of the disease based on Standard guidelines. This allows for steroid taper. 80% of subjects demonstrated reduction in 17-OHP (see FIG. 3). 50% of subjects demonstrated more than 25% reduction in 17-OHP. 50% of subjects were within the guideline range (1200 ng/dL) after treatment. Compound 1 attenuates morning rise in A4 which indicates an ability to control excess androgen production and associated symptoms (see FIG. 4). 100% of subjects demonstrated reduction in Androstenedione (at various doses). 60% of subjects demonstrated more than 25% reduction in Androstenedione. 50% of subjects were within normal reference range after treatment.

### Example 5

Formulation Development

The objectives of this experiment was to (1) evaluate different formulations to obtain a Compound 1 200 mg immediate release core tablet; (2) evaluate dissolution profiles of Compound 1 tablets in wet granulation process and in granulation with Gelucire 48/16 and/or Vitamin E TPGS; and (3) evaluate dissolution of the tablets/capsules in various bio-relevant media and sink conditions to compare dissolution of API in capsule.

### Experimental Outcomes

The formulation of Compound 1 tablets were performed in two phases.

In the first phase of manufacturing, two immediate release trial formulations were manufactured. The first trial involved granulation of Compound 1 using Gelucire (10%) and included fillers and disintegrants. No surfactant was used in his formulation. The granules were final blended and compressed at a tablet weight of 500 mg. No issues were observed in granulation and compression. The second trial involved wet granulation with HPC as the binder with fillers and disintegrants. Sodium lauryl sulphate was used as a surfactant at a concentration of 1%. The granules were softer when compared to the first trial and the final blend had poor flow. The filler in the extra granular portion was increased to improve flow and weight variation during compression. The tablets were compressed at 600 mg tablet weight. The first and second trial batches were tested for dissolution in different bio-relevant media—SGF (simulated gastric fluid), SIF (simulated intestinal fluid), FaSSIF (fasted state simulated intestinal fluid) and FeSSIF (fed state simulated intestinal fluid).

In the second phase of manufacturing, two additional formulations with Gelucire and Vitamin E TPGS at a percentage of 8% and with high concentration of sodium lauryl sulphate were manufactured. The percentage of the surfactant was increased to 7%. The tablets were compressed at 570 mg tablet weight. No issues were observed in the manufacturing process. Further development included manufacturing with high concentration of Gelucire (25%). Due to the high concentration of the Gelucire in the formulation, the granules could not be compressed and were manually filed into size 0 capsules.

Further development of wet granulation process was recommended as the flow was poor and particle size of granules was not optimum. The granules from the wet granulation formulation also showed weight variation during compression.

The release in SIF and FaSSIF was very low, ranging from between 0.5% to 3% at 60 minutes for the Gelucire and HPC formulations. The release in SGF and FeSSIF was higher and ranged from 11-16% in 60 minutes. The higher release in SGF and FeSSIF may have been due to the presence of surfactants in the media. However, the dissolution in the bio-relevant media did not show improvement with the lipid excipients or high concentration of surfactants. The release in SIF and FeSSIF was below 2%, in SGF approximately 15% and 10% in FeSSIR at 60 minutes. Dissolution showed no improvement with high concentration of Gelucire in the capsule formulation. A comparison of the dissolution of API in capsule and the different formulations did not show any improvement in dissolution. An animal pK study was also prformed with the wet granulation formulation and the Gelucire formulation. The dissolution and pK studies indicate that the current formulation approach may not be suitable to improve bioavailability of Compound 1 or minimize the food effect. A different formulation approach was recommended for Compound 1 in Phase 2/3 studies.

### Example 6

Preliminary Tablet Formulation

The objective of the general experiments were to develop, manufacture and commercialize a small tablet or capsule for a drug substance that is insoluble (solubility less than 0.002 mg/ml in water and at all physiological pHs ranging from 1.2 to 7.5) and a dose strength up to 200 mg. Tablet should be immediately released in a dissolution medium using USP apparatus I or II.

### Success Criteria

1. A tablet size less than 400 mg for a dose strength of 200 mg

2. More than 70% dissolved in 60 minutes in conventional dissolution media.

### General Manufacturing Procedure

The relevant excipients were manually sieved and then dry mixed. A binder solution was added and the mixture subjected to wet granulation. The granules were then wet milled and dried. The resulting granules were dry milled and added to other relevant common granules and excipients. The mixture was then compressed into tablets to a target tablet weight.

A granulation was developed with a minimum amount and number of excipients. The granulation consisted of at least 90% Compound 1 with a surfactant and a binder and in some cases, a super-disintegrant. After granulation was successful, other excipients were added to enable compression. These excipients included a compression aid, a lubricant, an anti-tacking agent and a super-disintegrant.

Two blends were granulated. One blend had 95% Compound 1, 1% sodium lauryl sulfate, and 4% hydroxypropylcellulose (HPC-EXF). The second blend had 91% Compound 1, 1% sodium lauryl sulfate (surfactant and wetting agent), 6% polyvinylpyrrolidone (PVP (water soluble binder) and 2% croscarmellose sodium (Ac-di-sol) (water-swellable super-disintegrant).

The granulation consisting of HPC-EXF (binder) was further blended with Silicon dioxide (SiO₂)(flow aid and anti-tacking agent), ac-di-sol (water swellable super-disintegrant) and magnesium stearate (lubricant). Different levels of Microcrystalline Cellulose were added to the tablets (0, 10 and 20% levels). All three formulations (90%, 81% and 71% DL) dissolved quickly, and there were no significant changes during accelerated stability in open dish conditions. The granulation with PVP as a binder was repeated three times. The first granulation consisted of 93% Compound 1, 6% PVP and 1% SLS. This formulation demonstrated inferior manufacturing features (granular flow) however the tablets had acceptable dissolution. No further stability was conducted.

The second granulation (identical to first) had acceptable manufacturing features (good flow and compression) however dissolution was somewhat slower and there were significant changes during on stability in very stressful conditions.

The third granulation consisted of 91% Compound 1, 6% PVP, 1% SLS and 2% Ac-di-sol as intra-granular super-disintegrant. The granulation was further blended with SiO₂ (flow aid and anti-tacking agent), ac-di-sol (water swellable disintegrant) and magnesium stearate (lubricant). Different levels of MCC were added to the tablets (0, 10 and 20% levels). All three formulations (86%, 77% AND 68% DL) dissolved quickly.

This demonstrated a successful tablet of Compound 1 with water solubility less than 0.002 mg/ml, while maintaining a drug load above 50% and resulting in acceptable dissolution in a conventional dissolution media (1% SLS and USP apparatus II). The tablet was manufactured using a conventional high shear wet granulation method.

TABLE 5
Summary of Compound 1 solubility in different aqueous
media (n = 2, 24 hours at room temperature)

| | | | Standard | |
|---|---|---|---|---|
| | Equilibrium | Solubility | Deviation | |
| Aqueous media | pH | mg/mL | (n = 2) | Description |
| Water | 7.78 | 0.0003 | 0.0000 | insoluble |
| 0.1N HCl | 1.06 | 0.0018 | 0.0000 | insoluble |
| 0.01N HCl | 2.06 | 0.0004 | 0.0000 | Insoluble |
| 0.001N HCl | 3.36 | 0.0003 | 0.0000 | Insoluble |
| pH 4.5 Acetate | 4.65 | 0.0005 | 0.0000 | Insoluble |
| (USP) | | | | |
| pH 6.0 Phosphate | 6.08 | 0.0002 | 0.0000 | Insoluble |
| (USP) | | | | |
| pH 7.5 Phosphate | 7.57 | 0.0006 | 0.0000 | Insoluble |
| (USP) | | | | |
| 0.1N NaOH | 11.65 | 0.0002 | 0.0000 | Insoluble |
| SGF | 2.25 | 0.0204 | 0.0001 | Insoluble |
| FaSSIF | 6.73 | 0.0036 | 0.0000 | Insoluble |
| FaSSIF | 5.16 | 0.0104 | 0.0000 | insoluble |

### Example 7

Tablet Formulation A

Three formulations were prepared using wet granulations with common granules and PVP K30 as a binder (see Table 6). The granules were diluted with MCC PH102 to reach88%,79%% and 70% drug loads respectively.

TABLE 6
Manufacturing Formulae A

| | A-1 | | A-2 | | A-3 | |
|---|---|---|---|---|---|---|
| Material | % w/w | mg/tablet | % w/w | mg/tablet | % w/w | mg/tablet |

Common Granular

| | | | | | | |
|---|---|---|---|---|---|---|
| Compound 1 | 93 | 200.00 | 93 | 200.00 | 93 | 200.00 |
| Povidone Compendial [plasdoneK- | 6 | 12.90 | 6 | 12.90 | 6 | 12.90 |
| 29/32] | | | | | | |
| Kolliphor ® SLS fine | 1 | 2.15 | 1 | 2.15 | 1 | 2.15 |

Extra Granular

| | | | | | | |
|---|---|---|---|---|---|---|
| Common granules | 95 | 215.05 | 85 | 215.05 | 75 | 215.05 |
| Microcrystalline Cellulose PH102 | — | 0.00 | 10 | 25.30 | 20 | 57.35 |
| Croscarmellose Sodium, Compendial | 3 | 6.79 | 3 | 7.59 | 3 | 8.60 |
| Silica Dioxide (Aerosil200Pharma) | 1 | 2.26 | 1 | 2.53 | 1 | 2.87 |
| LIGAMEDMF-2-V Magnesium Stearate | 1 | 2.26 | 1 | 2.53 | 1 | 2.87 |
| Target Tablet Weight (mg) | | 226.37 | | 253.00 | | 286.73 |

Compound 1, Povidone and sodium lauryl sulfate were mixed to produce a dry mixture. Water was added to the dry mixture and subjected to a wet granulation at an impeller speed of about 550-600 rpm. The wet granules were then sifted and dried. The dry granules were dry-sieved after which the granules were compressed into tablets.

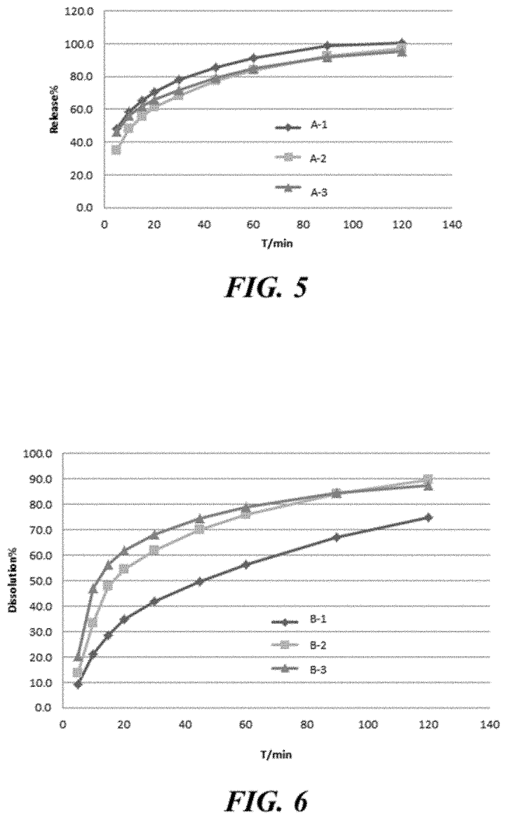

In process control tests, each tablet was satisfactory. Dissolution results of A-1 and A-3 showed similar curves whereas A-2 was slightly slower than the other two in the first few points (see table 7 and FIG. 5).

TABLE 7
Dissolution for Manufacturing Formulae A1
Dissolution in 0.1N HCl + 1.0% SDS, media at 50 rpm
and 900 ml using USP-II (Paddle)

| Time points | | | |
|---|---|---|---|
| (Minutes) | A-1 | A-2 | A-3 |
| 5 minutes | 48.4 | 35.0 | 46.7 |
| 10 minutes | 58.3 | 48.2 | 56.4 |
| 15 minutes | 65.2 | 55.9 | 62.2 |
| 20 minutes | 70.4 | 61.3 | 65.9 |
| 30 minutes | 78.1 | 68.3 | 71.9 |
| 45 minutes | 85.6 | 77.4 | 79.3 |
| 60 minutes | 91.5 | 84.1 | 85.1 |
| 90 minutes | 98.5 | 92.6 | 91.9 |
| 120 minutes | 100.5 | 97.2 | 95.6 |

### Example 8

Tablet Formulation B

Three formulations were prepared using wet granulation with common granules and PVP K30 as a binder (see Table 8). The granules were diluent with MCC PH102 with 88%%, 79% and 70% drug loads respectively.

TABLE 8
Manufacturing Formulae B

| | B-1 | | B-2 | | B-3 | |
|---|---|---|---|---|---|---|
| Material | % w/w | mg/tablet | % w/w | mg/tablet | % w/w | mg/tablet |

Common Granular

| | | | | | | |
|---|---|---|---|---|---|---|
| Compound 1 | 93 | 200.00 | 93 | 200.00 | 93 | 200.00 |
| Povidone Compendial [plasdoneK- | 6 | 12.90 | 6 | 12.90 | 6 | 12.90 |
| 29/32] | | | | | | |
| Kolliphor ® SLS fine | 1 | 2.15 | 1 | 2.15 | 1 | 2.15 |

Extra Granular

| | | | | | | |
|---|---|---|---|---|---|---|
| Common granules | 95 | 215.05 | 85 | 215.05 | 75 | 215.05 |
| Microcrystalline Cellulose PH102 | — | 0.00 | 10 | 25.30 | 20 | 57.35 |
| Croscarmellose Sodium, Compendial | 3 | 6.79 | 3 | 7.59 | 3 | 8.60 |
| Silica Dioxide (Aerosil200Pharma) | 1 | 2.26 | 1 | 2.53 | 1 | 2.87 |
| LIGAMEDMF-2-V Magnesium Stearate | 1 | 2.26 | 1 | 2.53 | 1 | 2.87 |
| Target Tablet Weight (mg) | | 226.37 | | 253.00 | | 286.73 |

Compound 1, Povidone and sodium lauryl sulfate were mixed to produce a dry mixture. Water was added to the dry mixture and subjected to a wet granulation at an impeller speed of about 600-610 rpm. The wet granules were then sifted and dried. The dry granules were dry-sieved after which the granules were compressed into tablets.

Dissolution results showed slower dissolution than previous tablets which may be caused by more granules and more hardness (see table 9 and FIG. 6).

TABLE 9
Dissolution for Manufacturing Formulae B
Dissolution in 0.1N HCl + 1.0% SDS, media at 50 rpm
and 900 ml using USP-II (Paddle)

| Time points | | | |
|---|---|---|---|
| (Minutes) | B-1 | B-2 | B-3 |
| 5 minutes | 9.4 | 13.7 | 20.4 |
| 10 minutes | 21.2 | 33.3 | 47.0 |
| 15 minutes | 28.6 | 47.8 | 56.4 |
| 20 minutes | 34.7 | 54.3 | 61.9 |
| 30 minutes | 41.8 | 62.0 | 68.2 |
| 45 minutes | 49.5 | 70.1 | 74.4 |
| 60 minutes | 56.3 | 75.8 | 78.8 |
| 90 minutes | 66.9 | 84.0 | 84.3 |
| 120 minutes | 74.8 | 89.5 | 87.4 |

### Example 9

Tablet Formulation C

Three formulations were prepared using wet granulation with common granules, PVP K30 as a binder, and 2% Croscarmellose Sodium Intragranular (see Table 10).

TABLE 10
Manufacturing Formulae C

| | C-1 | | C-2 | | C-3 | |
|---|---|---|---|---|---|---|
| Material Name | % w/w | Mg/tablet | % w/w | Mg/tablet | % w/w | Mg/tablet |

Common Granular

| | | | | | | |
|---|---|---|---|---|---|---|
| Compound 1 | 91 | 200 | 91 | 200 | 91 | 200 |
| Povidone Compendial [plasdoneK-29/32] | 6 | 13.2 | 6 | 13.2 | 6 | 13.2 |
| Kolliphor ® SLS Fine | 1 | 2.2 | 1 | 2.2 | 1 | 2.2 |
| Croscarmellose Sodium, Compendial | 2 | 4.4 | 2 | 4.4 | 2 | 4.4 |

Extra Granular

| | | | | | | |
|---|---|---|---|---|---|---|
| Common granules | 95 | 219.78 | 85 | 219.78 | 75 | 219.78 |
| Microcrystalline Cellulose PH102 | — | — | 10 | 25.86 | 20 | 58.61 |
| Croscarmellose Sodium, Compendial | 3 | 6.94 | 3 | 7.76 | 3 | 8.79 |
| Silica Dioxide(Aerosil200Pharma) | 1 | 2.31 | 1 | 2.59 | 1 | 2.93 |
| LIGAMEDMF-2-V Magnesium Stearate | 1 | 2.31 | 1 | 2.59 | 1 | 2.93 |
| Target Tablet Weight (mg) | | 231.36 | | 258.60 | | 293.06 |

Compound 1, Povidone, croscarmellose sodium and sodium lauryl sulfate were mixed to produce a dry mixture. Water was added to the dry mixture and subjected to a wet granulation at an impeller speed of about 600-610 rpm. The wet granules were then sifted and dried. The dry granules were dry-sieved after which the granules were compressed into tablets.

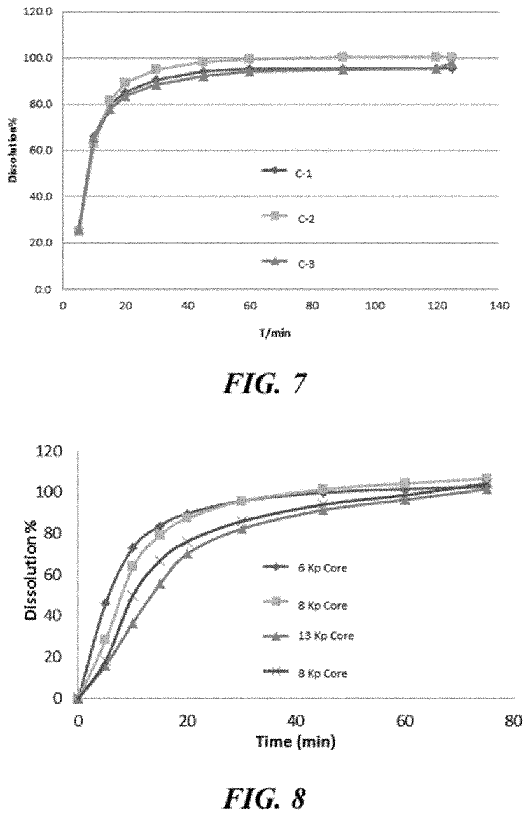

Dissolution results showed faster dissolution than other tablets which may be caused by the additional quantity of croscarmellose sodium that was added inside the granules (see Table 11 and FIG. 7).

TABLE 11
Dissolution for Manufacturing Formulae C
Dissolution in 0.1N HCl + 1.0% SDS, media at 50 rpm
and 900 ml using USP-II (Paddle)

| Time points | | | |
|---|---|---|---|
| (Minutes) | C-1 | C-2 | C-3 |
| 5 minutes | 26.3 | 25.1 | 26.2 |
| 10 minutes | 66.3 | 62.7 | 65.8 |
| 15 minutes | 79.9 | 81.7 | 77.8 |
| 20 minutes | 85.4 | 89.3 | 83.6 |
| 30 minutes | 90.6 | 94.9 | 88.5 |
| 45 minutes | 94.2 | 98.3 | 92.4 |
| 60 minutes | 95.4 | 99.6 | 94.1 |
| 90 minutes | 95.3 | 100.2 | 95.0 |
| 120 minutes | 95.5 | 100.4 | 95.7 |
| 250 RPM/5 min | 95.6 | 100.5 | 98.0 |

### Example 10

Tablet Formulation D

Two formulations were prepared using wet granulation (see Table 12).

TABLE 12
Manufacturing Formulae D

| | D-1 | | | D-2 | | |
|---|---|---|---|---|---|---|
| | | | Batch | | | Batch |
| Material Name | % w/w | mg/tablet | formula | % w/w | Mg/tablet | formula |
| Compound 1 | 71.25 | 200.000 | 570.00 | 77.35 | 200.000 | 546.00 |
| Klucel(TM)EXF PHARM Hydroxypropyl | 3.00 | 8.421 | 24.00 | — | — | — |
| cellulose | | | | | | |
| Povidone Compendial [plasdoneK-29/32] | — | — | — | 5.10 | 13.187 | 36.00 |
| Kolliphor ® SLS Fine | 0.75 | 2.105 | 6.00 | 0.85 | 2.198 | 6.00 |
| Croscarmellose Sodium, Compendial | — | — | — | 1.70 | 4.396 | 12.00 |

Extra granular material

| | | | | | | |
|---|---|---|---|---|---|---|
| Microcrystalline Cellulose PH102 | 20.00 | 56.140 | 160.00 | 10.00 | 25.856 | 70.59 |
| Croscarmellose Sodium, Compendial | 3.00 | 8.421 | 24.00 | 3.00 | 7.757 | 21.18 |
| Silica Dioxide (Aerosil200 Pharma) | 1.00 | 2.807 | 8.00 | 1.00 | 2.586 | 7.06 |
| LIGAMEDMF-2-V Magnesium Stearate | 1.00 | 2.807 | 8.00 | 1.00 | 2.586 | 7.06 |
| Target Tablet Weight (mg) | | 280.701 | 800.00 | | 258.566 | 705.89 |

In Formulation D-1, Compound 1, Povidone, croscarmellose sodium and sodium lauryl sulfate were mixed to produce a dry mixture. In formulation, D-2 Compound 1, HPC and sodium lauryl sulfate were mixed to produce a dry mixture. Water was added to both dry mixtures and subjected to a wet granulation at an impeller speed of about 600-610 rpm. The wet granules were then sifted and dried. The dry granules were dry-sieved after which the granules were compressed into tablets.

The release profile of Formulation D-1 and D2 were similar to previous formulations with no significant changes (see Table 13).

TABLE 13
Dissolution for Manufacturing Formulae C
Dissolution in 0.1N HCl + 1.0% SDS, media at 50 rpm
and 900 ml using USP-II (Paddle)

| Time points | | |
|---|---|---|
| (Minutes) | D-1 | D-2 |
| 5 minutes | 18.7 | 30.6 |
| 10 minutes | 43.8 | 72.1 |
| 15 minutes | 56.9 | 85.1 |
| 20 minutes | 65.2 | 91.5 |
| 30 minutes | 73.9 | 96.4 |
| 45 minutes | 81.9 | 99.9 |
| 60 minutes | 87.1 | 101.3 |
| 90 minutes | 92.4 | 102.3 |
| 120 minutes | 95.6 | — |
| 250 RPM/5 min | 99.4 | 100.9 |

Both D-1 and D-2 formulations had a faster release profile in SGF media than the powder in capsule formulation (see Table 14).

TABLE 14
Dissolution for Manufacturing Formulae C in SGF media
Dissolution in SGF, media at 60 rpm and 900 ml using USP-II (Paddle)

| Time points | | | Capsule with |
|---|---|---|---|
| (Minutes) | D-1 | D-2 | Compound 1 |
| 5 minutes | 10.8 | 7.0 | 0.3 |
| 10 minutes | 18.4 | 11.0 | 1.9 |
| 15 minutes | 21.3 | 11.8 | 4.2 |
| 20 minutes | 23.4 | 12.2 | 6.5 |
| 30 minutes | 24.4 | 12.4 | 9.4 |
| 45 minutes | 26.1 | 12.8 | 13.6 |
| 60 minutes | 25.7 | 13.0 | 15.1 |
| 90 minutes | 27.9 | 13.1 | 15.5 |
| 120 minutes | 28.2 | 13.2 | 15.2 |
| 250 RPM/5 min | 28.0 | 13.2 | 15.8 |

### Example 11

Tablet Formulation E

A formulation was prepared using wet granulation (see Table 15).

TABLE 15
Manufacturing Formulae E

| | E-1 | |
|---|---|---|
| Material Name | % w/w | Mg/tablet |
| Compound 1 | 71.25 | 100 |
| Klucel (TM)EXF PHARM Hydroxypropyl cellulose | 3 | 4.21 |
| Kolliphor ® SLS Fine | 0.75 | 1.05 |
| Extragranular | | |
| Microcrystalline Cellulose PH102 | 20 | 28.07 |
| Croscarmellose Sodium, Compendial | 3 | 4.21 |
| Silica Dioxide(Aerosil200Pharma) | 1 | 1.40 |
| LIGAMEDMF-2-V Magnesium Stearate | 1 | 1.40 |
| Total Weight | | 140.35 |
| OPADRY 8542129 Yellow | ~3 | 4.21 |

Compound 1, Sodium lauryl sulfate and HPC EXF were mixed to produce a dry mixture. Water was added to the dry mixture and subjected to a wet granulation at an impeller speed of about 550-590 rpm. The wet granules were then sifted and dried. The dry granules were dry-sieved after which the granules were compressed into tablets.

For F-1, tablet hardness had an impact on tablet dissolution. As hardness increased, tablet dissolution seemed to be slower especially at initial time points (see FIG. 8).

### Example 12

Tablet Formulation F

A formulation was prepared using wet granulation (see Table 16).

TABLE 16
Manufacturing Formulae F

| | F-1 | |
|---|---|---|
| Material Name | % w/w | Mg/tablet |
| Compound 1 | 71.25 | 50 |
| Klucel (TM)EXF PHARM Hydroxypropyl cellulose | 3 | 2.105 |
| Kolliphor ® SLS Fine | 0.75 | 0.526 |
| Extragranular | | |
| Microcrystalline Cellulose PH102 | 20 | 14.035 |
| Croscarmellose Sodium, Compendial | 3 | 2.105 |
| Silica Dioxide(Aerosil200Pharma) | 1 | 0.702 |
| LIGAMEDMF-2-V Magnesium Stearate | 1 | 0.702 |
| Total Weight | 100 | 70.175 |
| OPADRY 8542129 Yellow | ~3 | 2.11 |

Compound 1, sodium lauryl sulfate and HPC EXF were mixed to produce a dry mixture. Water was added to the dry mixture and subjected to a wet granulation at an impeller speed of about 550-590 rpm. The wet granules were then sifted and dried. The dry granules were dry-sieved after which the granules were compressed into tablets.

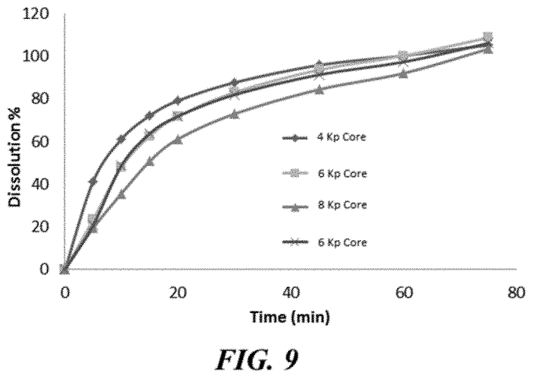

For G-1, tablet hardness had an impact on tablet dissolution. As hardness increased, tablet dissolution seemed to be slower especially at initial time points (see FIG. 9).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula I: 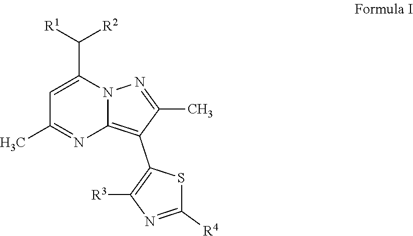
or a pharmaceutically acceptable salt thereof, wherein
R¹ and R² are independently ethyl or n-propyl;
R³ is H, Cl, Br, methyl, trifluoromethyl or methoxy;
R⁴ is H, Br, R^{a}R^{b}N—, methoxymethyl, n-butyl, acetamido, pyridin-4-yl, morpholin-4-yl, 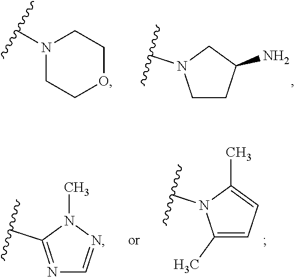
R^{a} and R^{b} are independently hydrogen, C₁-C₃ alkyl, H₂NCH₂CH₂—,
(CH₃)₃COC(O)NHCH₂CH₂—, or CH₃CH₂CH₂NHCH₂CH₂;
wherein the compound of Formula I contains at least one deuterium atom; and
wherein the deuterium enrichment content of the compound of Formula I is at least about 1%.

2. The compound of claim 1, or a pharmaceutically acceptable salt thereof, wherein R³ is Cl, Br, methyl or trifluoromethyl.

3. The compound of claim 1, or a pharmaceutically acceptable salt thereof, wherein R³ is Cl or Br.

4. The compound of claim 1, or a pharmaceutically acceptable salt thereof, wherein R⁴ is R^{a}R^{b}N—, pyridin-4-yl, morpholin-4-yl, or

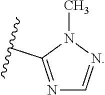 [SMILES: *c1ncnn1C]

5. The compound of claim 1, or a pharmaceutically acceptable salt thereof, wherein R⁴ is morpholin-4-yl or

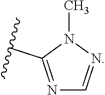 [SMILES: *c1ncnn1C]

6. The compound of claim 1, or a pharmaceutically acceptable salt thereof, wherein R⁴ is R^{a}R^{b}N— and R^{a} and R^{b} are independently C₁-C₃alkyl.

7. The compound of claim 1, or a pharmaceutically acceptable salt thereof, wherein the deuterium enrichment in the compound of Formula I is at least about 2%.

8. The compound of claim 1, or a pharmaceutically acceptable salt thereof, which is 3-[4-bromo-2-(2-methyl-2H-[1,2,4]triazol-3-yl)-thiazol-5-yl]-2,5-dimethyl-7-(1-propyl-butyl)-pyrazolo[1,5-a]pyrimidine, wherein the 7-(1-propyl-butyl)moiety comprises at least one deuterium.

9. The compound of claim 1, or a pharmaceutically acceptable salt thereof, which is 3-(4-bromo-2-(2-methyl-2H-[1,2,4]triazol-3-yl)-thiazol-5-yl)-7-(1-ethyl-propyl)-2,5-dimethyl-pyrazolo[1,5-a]pyrimidine, wherein the 7-(1-ethyl-propyl) moiety comprises at least one deuterium.

10. The compound of claim 1, or a pharmaceutically acceptable salt thereof, which is 3-(4-chloro-2-morpholin-4-yl-thiazol-5-yl)-7-(1-ethyl-propyl)-2,5-dimethy-1-pyrazolo[1,5-a]pyrimidine wherein the 7-(1-ethyl-propyl) moiety comprises at least one deuterium.

11. The compound of claim 1, or a pharmaceutically acceptable salt thereof, which is 3-(4-chloro-2-(morpholin-4-yl)thiazol-5-yl)-7-(1-ethyl-propyl)-2,5-dimethylpyrazolo(1,5-a)pyrimidine or 4-(4-chloro-5-(2,5-dimethyl-7-(pentan-3-yl)pyrazolo[1,5-a]pyrimidin-3-yl)thiazol-2-yl)morpholine, wherein the 7-(1-ethyl-propyl) moiety comprises at least one deuterium.

12. A pharmaceutical composition comprising Compound 1: 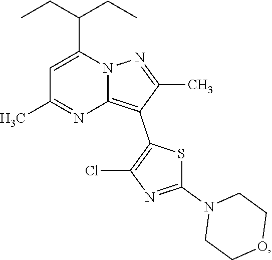 [SMILES: CCC(CC)c1cc(C)nc2c(-c3sc(N4CCOCC4)nc3Cl)c(C)nn12]
or a pharmaceutically acceptable salt or solvate thereof, and Compound 2: 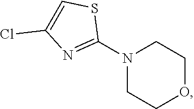 [SMILES: Clc1csc(N2CCOCC2)n1]
or a pharmaceutically acceptable salt or solvate thereof.

13. A pharmaceutical composition of claim 12, wherein the pharmaceutical composition comprises from about 0.5 ppm to about 5000 ppm of Compound 2, or a pharmaceutically acceptable salt or solvate thereof.

14. A pharmaceutical composition of claim 12, wherein the pharmaceutical composition comprises from about 0.00005 weight % to about 0.5 weight % of Compound 2, or a pharmaceutically acceptable salt or solvate thereof.

15. A pharmaceutical composition comprising Compound 1: 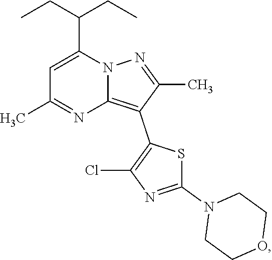 [SMILES: CCC(CC)c1cc(C)nc2c(-c3sc(N4CCOCC4)nc3Cl)c(C)nn12]
or a pharmaceutically acceptable salt or solvate thereof, which has a D₉₀ from about 1 μm to about 20 μm.

16. A pharmaceutical composition of claim 15, wherein Compound 1, or a pharmaceutically acceptable salt or solvate thereof has a D₉₀ from about 5 μm to about 15 μm.

17. A pharmaceutical composition of claim 16, wherein Compound 1, or a pharmaceutically acceptable salt or solvate thereof has a D₉₀ of about 6 μm.

18. A pharmaceutical composition of claim 16, wherein Compound 1, or a pharmaceutically acceptable salt or solvate thereof has a D₉₀ of about 7 μm.

19. A pharmaceutical composition of claim 1, wherein the pharmaceutical composition is in an oral dosage form.

20. A pharmaceutical composition of claim 19, wherein the oral dosage form is selected from the group consisting of a tablet, a capsule, a buccal tablet, a sub-lingual table, an orally-disintegrating tablet, a thin film, a liquid solution, a liquid suspension, a syrup, a powder, solid crystals, minitabs, coated pellets and sachets.
